(19) United States
(12) Patent Application Publication
(10) Pub. No.: US 2022/0073878 A1
(43) Pub. Date: Mar. 10, 2022

(54) A NOVEL CD16+ NATURAL KILLER CELL AND A METHOD OF CULTURING CD16+ NATURAL KILLER CELL

(71) Applicant: Acepodia Biotechnologies Ltd., New Taipei City (TW)

(72) Inventors: Zih-Fei Cheng, New Taipei City (TW); Chia-Yun Lee, New Taipei City (TW); Hao-Kang Li, New Taipei City (TW); Yan-Liang Lin, New Taipei City (TW); Ching-Wen Hsiao, Castro Valley, CA (US); Yan-Da Lai, New Taipei City (TW); Yu-Pei Cheng, New Taipei City (TW); Hsiu-Ping Yang, New Taipei City (TW); Shih-Chia Hsiao, New Taipei City (TW)

(73) Assignee: Acepodia Biotechnologies Ltd., New Taipei City (TW)

(21) Appl. No.: 17/423,044

(22) PCT Filed: Jan. 16, 2020
(86) PCT No.: PCT/US2020/013883

§ 371 Date: Jul. 14, 2021

Related U.S. Application Data
(60) Provisional application No. 62/794,193, filed on Jan. 18, 2019.

(51) Int. Cl. C12N 5/0783; C07K 14/735; A61K 35/17

(52) U.S. Cl. CPC C12N 5/0636; C07K 14/70535; C12N 2501/599; C12N 2500/34; A61K 35/17

(57) ABSTRACT

The present invention provides a human CD16⁺ natural killer cell line. This human CD16⁺ natural killer cell line does not include synthetic, genetically modified or deliberately delivered polynucleotide encoding the CD16 receptor and is a non-tumorigenic cell line. Therefore, this human CD16⁺ natural killer cell line might provide considerable long-term safety for disease treatment.

### FIELD OF THE INVENTION

This present invention relates to a CD16⁺ nature killer cell and a method of culturing CD16⁺ nature killer cell; more particularly relates to a non-transgenic and non-tumorigenic CD16⁺ killer cell line as well as a culture method capable of mass proliferating CD16⁺ natural killer cells and maintaining CD16 expression.

### BACKGROUND OF THE INVENTION

Natural killer (NK) cells are lymphocytes that constitute an important component of the innate immune system, and they are best appreciated for innate defense against viral infections and in tumor cell surveillance. In humans, NK cells are classically identified by the absence of the T cell receptor complex (CD3⁻) and presence of neural cell adhesion molecule (CD56⁺). There are two main NK cell subsets in human peripheral blood, wherein the majority (>90%) of peripheral blood NK cells are CD3⁻CD56^{dim}CD16⁺ NK cells and the minority (10%) of peripheral blood NK cells are CD3⁻CD56^{bright}CD16⁻ NK cells (Orange JS, 2013).

CD 16 receptor (FcγRIII; it is a receptor for the Fc region of IgG and can bind to the Fc portion of IgG antibodies) is necessary for Antibody-dependent cell cytotoxicity (ADCC) processes carried out by human NK cells. In human, the polynucleotide encoding the CD16 receptor is located on q arm of chromosome 1 at position 1q23.3. Human NK cells expressing CD16 receptor can kill various types of target cells such as cancer cells, tumor cells, and HIV-infected cells through ADCC processes (Rezvani K and Rouce RH, 2015; Littwitz-Salomon et al, 2016; Eileen Scully and Galit Alter, 2016). Take tumor cells as an instance, tumor cells expressing tumor-associated antigens (such as human epidermal growth factor receptor 2, refer to as HER2) can bind to endogenous IgG antibodies or clinically approved therapeutic IgG antibodies targeting the tumor-associated antigens (such as trastuzumab, rituximab, or cetuximab). Once the CD 16 receptors (IgG Fc receptor FcγRIII) expressed by a NK cell bind to the Fc region of the endogenous IgG antibodies or clinically approved therapeutic IgG antibodies, NK cell-mediated ADCC will be triggered and the NK cell will then release cytotoxic factors that cause the death of tumor cells (Rezvani K and Rouce RH, 2015).

There are two major cancer treatment methods by using CD16+NK cells. The first method includes the following steps: (a) obtain autologous or allogeneic blood; (b) isolate autologous or allogeneic primary CD16⁺ natural killer cells (primary CD16+NK cells) from autologous or allogeneic blood; (c) proliferate autologous or allogeneic primary CD16⁺ NK cells in vitro; and (d) inject proliferated autologous or allogeneic primary CD16⁺ NK cell back to the veins of a cancer patient, so that there will be enough CD16⁺ NK cells in the cancer patient to release cytotoxic factors that cause the death of cancer cells through ADCC process. However, due to the fact that primary CD16⁺ NK cell will age and even die after several weeks of short-term culture, it is necessary to continuously obtain primary CD16⁺ NK cell from autologous or allogeneic blood for long-term treatment. Moreover, studies have demonstrated that in all of the cultured cells which are obtained from culturing the high-purity CD16⁺ NK cell population (i.e., the amount of CD16⁺ NK cells by number are equal to or more than 99%) with conventional method for 4 days, there are only 10% of cells still express CD16. In other words, the current method of culturing CD16⁺ NK cells in vitro cannot make NK cell stably express CD16 after proliferation. Therefore, the aforesaid method not only has difficulty in retaining the source of primary CD16⁺ NK cells, but also lack of method capable of stably proliferating CD16⁺ NK cells in vitro. These problems often make it difficult for cancer patients to acquire sufficient number of CD16⁺ NK cells and it is difficult to carry out cancer treatment smoothly each time. Moreover, the aforesaid method also needs to face the problem of difficulty in controlling the efficacy caused by individual cell differences.

The second method involves a NK-92 cell line (Deposit number ATCC CRL-2407). NK-92 cell line is a CD16⁻ natural killer cell line, isolated from blood of a fifty-year-old Caucasian male suffering from malignant non-Hodgkin's lymphoma. The NK-92 cell line can be continuously subcultured without aging and death problem, and this NK-92 cell line is not tumorigenic to immune compromised mice. After irradiated with γ-ray, it is also not carcinogenic to allogeneic human subjects, and thus there is a certain degree of applicability. However, since the NK-92 cell line does not express the CD16 receptor, it is unable to destroy cancer cells through ADCC process. Therefore, the aforesaid second method requires the genetic transfer of the CD16 receptor gene into the NK-92 cell line via transgenic technology in order to obtain a transgenic CD16 NK-92 cell line capable of expressing CD16 receptor and exerting ADCC. Then, the NK-92 cell line transfected with CD16 is injected into the vein of the cancer patient; therefore, there are enough CD16⁺ natural killer cells in the cancer patient to release cytotoxic factors that cause the death of cancer cells through ADCC process. Unfortunately, the medical community and the general public are concerned about the long-term safety of transgenic immune cells in the human circulatory system. Hence, the development of the aforesaid method is limited to a considerable extent.

Consequently, there is still an urgent need for a non-transgenic, and non-tumorigenic cell line that can be subcultured continuously as well as a culture method that is capable of mass proliferating CD16⁺ NK while maintaining the expression of CD16.

### SUMMARY OF THE INVENTION

The present invention provides a natural killer cell line that can be continuously subcultured without the issue of aging or dying.

The second purpose of the present invention is to provide a natural killer cell that can still stably express CD16⁺ receptor after at least 3 months of proliferation.

Another purpose of the present invention is to provide a non-transgenic CD16⁺ natural killer cell line.

Another purpose of the present invention is to provide a CD16⁺ natural killer cell line that is not tumorigenic to immune compromised mice.

Another purpose of the present invention is to provide a CD16⁺ natural killer cell line that is not carcinogenic to an allogeneic human subject after irradiation with γ-ray.

Another purpose of the present invention is to provide a culture method for mass proliferating of CD16⁺ natural killer cells.

Another purpose of the present invention is to provide a culture method that enables CD16⁺ natural killer to stably express CD16 receptor after proliferation.

Another purpose of the present invention is to provide a composition substantially enriched in human CD16⁺ natural killer cells, wherein the number of the human CD16⁺ natural killer cells in the composition is at least 5×10⁵ and the human CD16⁺ natural killer cells are in an amount equal to or more than 5% by number, based on the total number of the cells in the composition as 100%; the human CD16⁺ natural killer cell having the following characteristics: retaining its capability to proliferate after subculture for at least 3 months.

The present invention provides a human natural killer cell having the following characteristics: (i) expressing a CD16 receptor; (ii) retaining its capability to proliferate after subculture for at least 3 months; and (iii) comprising an expressed polynucleotide sequence encoding the CD16 receptor, wherein the expressed polynucleotide sequence encoding the CD16 receptor is not synthetic, not genetically modified and/or not deliberately delivered into the cells.

Preferably, the human natural killer cell is capable of proliferating after subculture for at least 1 week, 2 weeks, 3 weeks, 1 month, 2 months, 3 months, 4 months, 5 months, 6 months, 7 months, 8 months, 9 months, 10 months, or 11 months.

Preferably, the human natural killer cell is capable of proliferating after subculture for at least 1 year, 2 years, or 3 years.

Preferably, the human natural killer cell is non-tumorigenic in an immune compromised mouse.

Preferably, the immune compromised mouse is a SCID/Beige, a NOD/SCID, a NSG, or a nude mouse.

The present invention further provides a composition substantially enriched in human CD16⁺ natural killer cells, wherein the number of the human CD16⁺ natural killer cells in the composition is at least 5×10⁵ and the human CD16⁺ natural killer cells are in an amount equal to or more than 5% by number, based on the total number of the cells in the composition as 100%; the human CD16⁺ natural killer cell having the following characteristics: (i) expressing a CD16 receptor, (ii) retaining its capability to proliferate after subculture for at least 3 months, and (iii) comprising an expressed polynucleotide sequence encoding the CD16 receptor, wherein the polynucleotide sequence encoding the CD16 receptor is not synthetic, not genetically modified and/or not deliberately delivered into the cells.

Preferably, the number of the human CD16⁺ natural killer cells in the composition is 5×10⁵-5×10⁹.

Preferably, the number of the human CD16⁺ natural killer cells in the composition is 1×10⁶, 1.1×10⁶, 5×10⁶, 5.1×10⁶, 1×10⁷, 1.1×10⁷, 5×10⁷, 5.1×10⁷, 1×10⁸, 1.1×10⁸, 5×10⁸, 5.1×10⁸, 1×10⁹, 1.1×10⁹, or 5×10⁹.

Preferably, the total amount of the human CD16⁺ natural killer cells is 5%-100%, based on the total number of the cells in the composition as 100%.

Preferably, the human CD16⁺ natural killer cells are in an amount equal to or more than 5%, 7%, 9%, 10%, 12%, 15%, 19%, 20%, 22%, 25%, 29%, 30%, 32%, 35%, 39%, 40%, 42%, 45%, 49%, 50%, 52%, 55%, 59%, 60%, 62%, 65%, 69%, 70%, 72%, 75%, 79%, 80%, 82%, 85%, 89%, or 95% by number, based on the total number of the cells in the composition as 100%.

Preferably, the CD16 receptor is a CD16a receptor or a CD16b receptor.

Preferably, the expressed polynucleotide sequence encoding the CD16a receptor or the CD16b receptor is not synthetic, not genetically modified and/or not deliberately delivered into cells.

A method of obtaining a composition substantially enriched in human CD16⁺ natural killer cells; the method comprising: (a) obtaining a population of human peripheral blood natural killer cells having the deposit number ATCC CRL-2047; (b) contacting the population of human peripheral blood natural killer cells with an antibody specific for a CD16 receptor; and (c) separating cells that are specifically bound by the antibody thereby obtaining the composition substantially enriched in human CD16⁺ natural killer cells; wherein the human CD16⁺ natural killer cell comprises an expressed polynucleotide encoding a CD16 receptor, and the expressed polynucleotide sequence encoding the CD16 receptor is not synthetic, not genetically modified and/or not deliberately delivered into the cells.

Preferably, the human CD16⁺ natural killer cells are capable of retaining their capability to proliferate after subculture for at least 3 months.

Preferably, the human CD16⁺ natural killer cells are capable of retaining their capability to proliferate after subculture for at least 1 week, 2 weeks, 3 weeks, 1 month, 2 months, 3 months, 4 months, 5 months, 6 months, 7 months, 8 months, 9 months, 10 months, or 11 months.

Preferably, the human CD16⁺ natural killer cells are capable of retaining their capability to proliferate after subculture for at least 1 year, 2 years, or 3 years.

Preferably, the expressed polynucleotide sequence encoding the CD16 receptor is not synthetic, not genetically modified and/or not deliberately delivered into the cells.

Preferably, in the composition, the human CD16⁺ natural killer cells are in an amount equal to or more than 80% by number, based on the total number of the cells in the composition as 100%.

Preferably, the human CD16⁺ natural killer cells are in an amount equal to or more than 5% by number, based on the total number of the cells in the composition as 100%.

Preferably, the human CD16⁺ natural killer cells are in an amount equal to or more than 50% by number, based on the total number of the cells in the composition as 100%.

Preferably, the concentration of the dissolved glucose in the culture medium is higher than 1500 mg/L.

Preferably, the culture medium is fully aerated, the concentration of the dissolved oxygen in the culture medium is maintained in a stable range, or the concentration of the dissolved glucose in the culture medium is 1500-5000 mg/L.

Preferably, the concentration of the dissolved glucose in the culture medium is 2500, 2501, 3500, 3501, 4000, or 4500 mg/L.

Preferably, the number of the human CD16⁺ natural killer cells in the composition is at least 5×10⁵, and the human CD16⁺ natural killer cells are in an amount equal to or more than 5% by number, based on the total number of the cells in the composition as 100%.

Preferably, the number of the human CD16⁺ natural killer cells in the composition is 5×10⁵-5×10⁹.

Preferably, the number of the human CD16⁺ natural killer cells in the composition is 1×10⁶, 1.1×10⁶, 5×10⁶, 1×10⁷, 1.1×10⁷, 5×10⁷, 5.1×10⁷, 1×10⁸, 1.1×10⁸, 5×10⁸, 5.1×10⁸, 1×10⁹, 1.1×10⁹, 5×10⁹, 1×10¹⁰, 1.1×10¹⁰, 5×10¹⁰, 1×10¹¹, 1.1×10¹¹, 5×10¹¹, 5.1×10¹¹, 1×10¹², 1.1×10¹², 5×10¹², 5.1×10¹², 1×10¹³, 1.1×10¹⁴, 5×10¹⁴, 1×10¹⁵, 1.1×10¹⁵, 5×10¹⁵, 1×10¹⁶, 1.1×10¹⁶, 5×10¹⁶, 5.1×10¹⁶, 1×10¹⁷, 1.1×10¹⁷, 5×10¹⁷, 5.1×10¹⁷, 1×10¹⁸, 1.1×10¹⁸, 5×10¹⁸, 1×10¹⁹, 1.1×10¹⁹, 5×10¹⁹, 1×10²⁰, 1.1×10²⁰, 5×10²⁰, 5.1×10²⁰, 1×10²¹, 1.1×10²¹, 5×10²¹, 5.1×10²¹, 1×10²², 1.1×10²², 5×10²², 1×10²³, 1.1×10²³, 5×10²³, 1×10²⁴, 1.1×10²⁴, 5×10²⁴, 5.1×10²⁴, 1×10²⁵, 1.1×10²⁵, 5×10²⁵, 5.1×10²⁵, 1×10²⁶, 1.1×10²⁶, 5×10²⁶, 1×10²⁷, 1.1×10²⁷, 5×10²⁷, 1×10²⁸, 1.1×10²⁸, 5×10²⁸, 5.1×10²⁸, 1×10²⁹, 1.1×10²⁹, 5×10²⁹, 5.1×10²⁹, 1×10³⁰, 1.1×10³⁰, 5×10³⁰, 1×10³¹, 1.1×10³¹, 5×10³¹, 1×10³², 1.1×10³², 5×10³², 5.1×10³², 1×10³³, 1.1×10³³, 5×10³³, 5.1×10³³, 1×10³⁴, 1.1×10³⁴, 5×10³⁴, 1×10³⁵, 1.1×10³⁵, 5×10³⁵, 1×10³⁶, 1.1×10³⁶, 5×10³⁶, 5.1×10³⁶, 1×10³⁷, 1.1×10³⁷, 5×10³⁷, 5.1×10³⁷, 1×10³⁸, 1.1×10³⁸, 5×10³⁸, 1×10³⁰, 1.1×10³⁰, 5×10³⁰, 5.1×10³⁰, 1×10⁴⁰, 1.1×10⁴⁰, 5×10⁴⁰. Preferably, the number of the human CD16⁺ natural killer cells in the composition is 1×10⁶-1×10⁴¹.

Preferably, the total number of the human CD16⁺ natural killer cells is 5%-100%, based on the total number of the cells in the composition as 100%.

Preferably, the human CD16⁺ natural killer cells are in an amount equal to or more than 5%, 7%, 9%, 10%, 12%, 15%, 19%, 20%, 22%, 25%, 29%, 30%, 32%, 35%, 39%, 40%, 42%, 45%, 49%, 50%, 52%, 55%, 59%, 60%, 62%, 65%, 69%, 70%, 72%, 75%, 79%, 80%, 82%, 85%, 89%, or 95% by number, based on the total number of the cells in the composition as 100%.

The present invention further provides a method of culturing and expanding human CD16⁺ natural killer cells; the method comprising: (x) in a container, contacting the human CD16⁺ natural killer cells with a culture medium comprising human platelet lysate and IL-2; and (y) culturing the cells for multiple days; wherein the human CD16⁺ natural killer cell comprises an expressed polynucleotide encoding a CD16 receptor and the expressed polynucleotide sequence encoding the CD16 receptor is not synthetic, not genetically modified and/or not deliberately delivered into the cells.

Preferably, the concentration of the dissolved glucose in the culture medium is higher than 1500 mg/L.

Preferably, the step (y) comprises substeps: (y1) culturing the cells for at least one day; and (y2) sub-culturing the cells for at least 3 months.

Preferably, wherein the step (y2) is to sub-culturing the cells for at least 1 week, 2 weeks, 3 weeks, 1 month, 2 months, 3 months, 4 months, 5 months, 6 months, 7 months, 8 months, 9 months, 10 months, or 11 months.

Preferably, wherein the step (y2) is to sub-culturing the cells for at least 1 year, 2 years, or 3 years.

Preferably, the human CD16⁺ natural killer cells are capable of retaining their capability to proliferate after subculture for at least 3 months.

Preferably, the human CD16⁺ natural killer cells are capable of retaining their capability to proliferate after subculture for at least 1 week, 2 weeks, 3 weeks, 1 month, 2 months, 3 months, 4 months, 5 months, 6 months, 7 months, 8 months, 9 months, 10 months, or 11 months.

Preferably, the human CD16⁺ natural killer cells are capable of retaining their capability to proliferate after subculture for at least 1 year, 2 years, or 3 years.

Preferably, the expressed polynucleotide sequence encoding the CD16 receptor is not synthetic, not genetically modified and/or not deliberately delivered in the cells.

Preferably, the human CD16⁺ natural killer cell expresses CD2 molecule (CD2⁺).

Preferably, the human CD16⁺ natural killer cell expresses NKp44, NKp46, NKG2D, or CD107a.

The present invention provides a human natural killer cell having the following characteristics: (i) expressing a CD16 receptor; (ii) retaining its capability to proliferate after subculture for at least 3 months; and (iii) comprising an expressed CD16A gene encoding the CD16 receptor, wherein the expressed CD16A gene is located on q arm of chromosome 1.

Preferably, the expressed CD16A gene is located on q arm of chromosome 1 at position 1q23.3.

The present invention provides a human natural killer cell having the following characteristics: (i) expressing a CD16 receptor; (ii) retaining its capability to proliferate after subculture for at least 3 months; and (iii) comprising an expressed CD16A gene encoding the CD16 receptor, wherein the expressed CD16A gene polynucleotide sequence is not synthetic, not genetically modified and/or not deliberately delivered into cells.

Preferably, the expressed CD16A gene polynucleotide sequence is not synthetic, not genetically modified and/or not deliberately delivered into the cells.

The present invention provides a human natural killer cell which is (A) deposited at NPMD having the deposit number NITE BP-03017; or (B) having the following characteristics:

i) expressing a CD16 receptor;

ii) retaining its capability to proliferate after subculture for at least 3 months; and

iii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to higher than 1, wherein the sequence of the CD16 F176F probe is SEQ ID NO: 11 and the sequence of the CD16 F176V probe is SEQ ID NO: 12.

Preferably, the CD16 receptor is a CD16a receptor or a CD16b receptor.

Preferably, an expressed polynucleotide encoding the CD16 receptor is located on q arm of chromosome 1 at position 1q23.3.

Preferably, the cell is non-tumorigenic in an immune compromised mouse.

Preferably, after being irradiated with γ-ray, the cell is non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the cell further comprises an inactive tumor suppressor gene or a mutated and highly expressed oncogene.

Preferably, the cell is capable of mediating an antibody-dependent cell cytotoxicity (ADCC) response, and the cell is a male cell.

Preferably, the cell further comprises at least an exogenous targeting unit complexed to the cell, wherein the exogenous targeting unit comprises a targeting moiety that is characterized in that: (a) it exhibits specific binding to a biological marker on a target cell; (b) it is not a nucleic acid; and (c) it is not produced by the cell.

Preferably, the exogenous targeting unit is complexed to the cell via an interaction between a first linker conjugated to the targeting moiety and a second linker conjugated to the cell.

Preferably, the first linker is a first polynucleotide, or the second linker is a second polynucleotide.

Preferably, the targeting moiety comprises an antigen-binding unit.

Preferably, the first polynucleotide comprises a single-stranded region.

Preferably, the first linker is a first polynucleotide, and the second linker is a second polynucleotide.

Preferably, the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

Preferably, the first linker comprises a first reactive group, and the second linker comprises a second reactive group, and wherein the cell is complexed to the targeting moiety via a covalent bond formed by a reaction between the second reactive group and the first reactive group.

Preferably, the targeting moiety comprises an antigen-binding unit.

Preferably, the second linker comprises a PEG region.

Preferably, the targeting moiety and the cell are separated by a length of 1 nm to 400 nm, or the targeting moiety and the cell are separated by a length of 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 60, 70, 80, 90, 100, 130, 170, 200, 230, 270, 300, 330, or 370 nm.

Preferably, the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, or MAGE.

Preferably, the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TW, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, activating NK cell receptors, and combinations thereof.

Preferably, the targeting moiety is conjugated to the first polynucleotide using a coupling group, wherein the coupling group is an NHS ester, other activated ester, an alkyl or acyl halide, a bifunctional crosslinker, or maleimide group.

Preferably, the first polynucleotide or second polynucleotide comprise a sequence selected from 20-mer poly-CA, 20-mer poly-GGTT, SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7, and SEQ ID NO:10.

Preferably, the binding affinity of the targeting moiety for the biological marker is less than 250 nM, or the binding affinity of the targeting moiety for the biological marker is 5 nM, 10 nM, 40 nM, 90 nM, 130 nM, or 170 nM.

Preferably, the length of the first polynucleotide or the length of the second polynucleotide are 4 nt to 500 nt. Preferably, the length of the first polynucleotide or the length of the second polynucleotide are 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 55, 60, 65, 70, 75, 80, 85, 90, 95, 100, 120, 160, 220, 300, 400, or 480 nt.

Preferably, the binding affinity between the first linker and the second linker is less thin 250 nM. Preferably, the binding affinity between the first linker and the second tinker is 5 nM, 10 nM, 40 nM, 90 nM, 130 nM, or 170 nM.

Preferably, the first linker or the second linker is conjugated to a native functional group of the targeting unit or a surface of the cell, wherein the native functional group is an amino acid, a sugar, or an amine.

Preferably, the targeting moiety is a peptide, protein, or aptamer.

The present invention provides a composition substantially enriched in human CD16 natural killer cells, wherein the number of the human CD16⁺ natural killer cells in the composition is at least 5×10⁵ and the human CD16⁺ natural killer cells are in an amount equal to or more than 5% by number; based on the total number of the cells in the composition as 100%; the human CD16⁺ natural killer cell is (A) deposited at NPMD having the deposit number NITE BP-03017; or (B) having the following characteristics:

i) expressing a CD16 receptor,

ii) retaining its capability to proliferate after subculture for at least 3 months, and

iii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

Preferably, the CD16 receptor is a CD16a receptor or a CD16b receptor.

Preferably, a polynucleotide encoding the CD16 receptor is located on q arm of chromosome 1 at position 1q23.3.

Preferably, the human CD16⁺ natural killer cells are non-tumorigenic in an immune compromised mouse.

Preferably, after being irradiated with γ-ray, the human CD16⁺ natural killer cells are non-tumorigenic in an allogeneic subject.

Preferably, a polynucleotide encoding the CD16 receptor comprises a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human CD16⁺ natural killer cell further comprises an inactive tumor suppressor gene or a mutated and highly expressed oncogene.

Preferably, the human CD16⁺ natural killer cell is capable of mediating an antibody-dependent cell cytotoxicity (ADCC) response, and the cell is a male cell.

Preferably, the human CD16⁺ natural killer cell further comprises at least an exogenous targeting unit complexed to the human CD16⁺ natural killer cell, wherein the exogenous targeting unit comprises a targeting moiety that is characterized in that: (a) it exhibits specific binding to a biological marker on a target cell; (b) it is not a nucleic acid; and (c) it is not produced by the human CD16⁺ natural killer cell.

Preferably, the exogenous targeting unit is complexed to the human CD16⁺ natural killer cell via an interaction between a first linker conjugated to the targeting moiety and a second linker conjugated to the human CD16⁺ natural killer cell.

Preferably, the first linker is a first polynucleotide, or the second linker is a second polynucleotide.

Preferably, the targeting moiety comprises an antigen-binding unit.

Preferably, the first polynucleotide comprises a single-stranded region.

Preferably, the first linker is a first polynucleotide, and the second linker is a second polynucleotide.

Preferably, the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

Preferably, the first linker comprises a first reactive group, and the second linker comprises a second reactive group, and wherein the human CD16⁺ natural killer cell is complexed to the targeting moiety via a covalent bond formed by a reaction between the second reactive group and the first reactive group.

Preferably, the targeting moiety comprises an antigen-binding unit.

Preferably, the second linker comprises a PEG region.

Preferably, the targeting moiety and the human CD16⁺ natural killer cells are separated by a length of 1 nm to 400 nm.

Preferably, the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, or MAGE.

Preferably, the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, activating NK cell receptors, and combinations thereof.

Preferably, the targeting moiety is conjugated to the first polynucleotide using a coupling group, wherein the coupling group is an NHS ester, other activated ester, an alkyl or acyl halide, a bifunctional crosslinker, or maleimide group.

Preferably, the first polynucleotide or second polynucleotide comprise a sequence selected from 20-mer poly-CA, 20-mer poly-GGTT, SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7, and SEQ ID NO:10.

Preferably, the binding affinity of the targeting moiety for the biological marker is less than 250 nM.

Preferably, the length of the first polynucleotide or the length of the second polynucleotide are 4 nt to 500 nt.

Preferably, the binding affinity between the first linker and the second linker is less than 250 nM.

Preferably, the first linker or the second linker is conjugated to a native functional group of the targeting unit or a surface of the human CD16⁺ natural killer cell, wherein the native functional group is an amino acid, a sugar, or an amine.

Preferably, the targeting moiety is a peptide, protein, or aptamer.

The present invention provides a method of obtaining a composition substantially enriched in human CD16⁺ natural killer cells; the method comprising: (a) obtaining a population of human peripheral blood natural killer cells having the deposit number ATCC CRL-2407; (b) contacting the population of human peripheral blood natural killer cells with an antibody specific for a CD16 receptor; and (c) separating cells that are specifically bound by the antibody thereby obtaining the composition substantially enriched in human CD16⁺ natural killer cells; wherein the human CD16⁺ natural killer cell is: (A) deposited at NPMD having the deposit number NITE BP-03017; or (B) having the following characteristics:

i) expressing a CD16 receptor, and

ii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

Preferably, the antibody is specific for at least one of a CD16a receptor and a CD16b receptor.

Preferably, the human CD16⁺ natural killer cells are capable of retaining their capability to proliferate after subculture for at least 1 week.

Preferably, an expressed polynucleotide encoding the CD16 receptor is located on q arm of chromosome 1 at position 1q23.3.

Preferably, the human CD16⁺ natural killer cells are non-tumorigenic in an immune compromised mouse.

Preferably, after being irradiated with γ-ray, the human CD16⁺ natural killer cells are non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human CD16⁺ natural killer cell further comprises an inactive tumor suppressor gene or a mutated and highly expressed oncogene.

Preferably, in the composition, the human CD16⁺ natural killer cells are in an amount equal to or more than 5% by number, based on the total number of the cells in the composition as 100%.

Preferably, the human CD16⁺ natural killer cells are capable of mediating an antibody-dependent cell cytotoxicity (ADCC) response, and the human CD16⁺ natural killer cells are male cells.

Preferably, the step (c) comprises substeps: (c1) separating cells that are specifically bound by the antibody; (c2) in a container, contacting the cells that are specifically bound by the antibody with a culture medium comprising human platelet lysate and IL-2; and (c3) culturing the cells for multiple days thereby obtaining the composition substantially enriched in human CD16⁺ natural killer cells.

Preferably, the container is G-Rex culture devices.

Preferably, the container comprises a bottom for seeding cells, and the bottom is air-permeable and water-impermeable.

Preferably, the concentration of the dissolved glucose in the culture medium is 1500-5000 mg/L.

Preferably, the number of the human CD16⁺ natural killer cells in the composition is at least 5×10⁵, and the human CD16⁺ natural killer cells are in an amount equal to or more than 5% by number, based on the total number of the cells in the composition as 100%.

The present invention provides a method of culturing and expanding human CD16⁺ natural killer cells; the method comprising (x) in a container, contacting the human CD16⁺ natural killer cells with a culture medium comprising 0.5-10 vol % human platelet lysate and 100-3000 IU/mLIL-2; and (y) culturing the cells for multiple days. Preferably, the culture medium comprised 1 vol %, 2 vol %, 3 vol %, 4 vol %, 5 vol %, 6 vol %, 7 vol %, 8 vol %, 9 vol %, 10 vol %, 11 vol %, 12 vol %, 13 vol %, 14 vol %, or 15 vol % human platelet lysate. Preferably, the culture medium comprised 0.5-20 vol % human platelet lysate. Preferably, the culture medium comprised 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 IU/mL IL-2.

Preferably, the container is G-Rex culture devices.

Preferably, the container comprises a bottom for seeding cells, and the bottom is air-permeable and water-impermeable.

Preferably, the concentration of the dissolved glucose in the culture medium is 1500-5000 mg/L.

Preferably, the step (y) comprises substeps: (y1) culturing the cells for at least one day; and (y2) sub-culturing the cells for at least 1 months.

Preferably, the human CD16⁺ natural killer cells are capable of retaining their capability to proliferate after subculture for at least 3 months.

Preferably the human CD16⁺ natural killer cell is: (A) deposited at NPMD having the deposit number NITE BP-03017; or (B) having the following characteristics:

i) expressing a CD16 receptor, and

ii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12

Preferably, the human CD16⁺ natural killer cells are non-tumorigenic in an immune compromised mouse.

Preferably, after being irradiated with γ-ray, the human CD16⁺ natural killer cells are non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human CD16⁺ natural killer cell further comprises an inactive tumor suppressor gene or a mutated and highly expressed oncogene.

The present invention provides a method of treating cancer, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of a human natural killer cell to a subject in need thereof; the human natural killer cell is (A) deposited at NPMD having the deposit number NITE BP-03017; or (B) having the following characteristics:

i) expressing a CD16 receptor,

ii) retaining its capability to proliferate after subculture for at least 3 months, and iii) x) not including synthetic, genetically modified and/or deliberately delivered polynucleotide encoding the CD16 receptor, or y) by using ddPCR system to analyze the genomic DNA of the human natural killer cell, the ratio of CD16 F176F probe-detectable DNA molecule to CD16 F176V probe-detectable DNA molecule is higher than 1, wherein the sequence of the CD16 F176F probe is SEQ ID NO: 11 and the sequence of the CD16 F176V probe is SEQ ID NO: 12.

Preferably, the human natural killer cell further comprises at least an exogenous targeting unit complexed to the human natural killer cell, wherein the exogenous targeting unit comprises a targeting moiety that is characterized in that: (a) it exhibits specific binding to a biological marker on a target cell; (b) it is not a nucleic acid; and (c) it is not produced by the human natural killer cell.

Preferably, the exogenous targeting unit is complexed to the human natural killer cell via an interaction between a first linker conjugated to the targeting moiety and a second linker conjugated to the human natural killer cell.

Preferably, the first linker is a first polynucleotide, or the second linker is a second polynucleotide.

Preferably, the targeting moiety comprises an antigen-binding unit.

Preferably, the first polynucleotide comprises a single-stranded region.

Preferably, the first linker is a first polynucleotide, and the second linker is a second polynucleotide.

Preferably, the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

Preferably, the first linker comprises a first reactive group, and the second linker comprises a second reactive group, and wherein the human natural killer cell is complexed to the targeting moiety via a covalent bond formed by a reaction between the second reactive group and the first reactive group.

Preferably, the targeting moiety comprises an antigen-binding unit.

Preferably, the second linker comprises a PEG region.

Preferably, the targeting moiety and the human natural killer cell are separated by a length of 1 nm to 400 mm.

Preferably, the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, or MAGE.

Preferably, the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, activating NK cell receptors, and combinations thereof.

Preferably, the targeting moiety is conjugated to the first polynucleotide using a coupling group, wherein the coupling group is an NHS ester, other activated ester, an alkyl or acyl halide, a bifunctional crosslinker, or maleimide group.

Preferably, the first polynucleotide or second polynucleotide comprise a sequence selected from 20-mer poly-CA, 20-mer poly-GGTT, SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7, and SEQ ID NO:10.

Preferably, the binding affinity of the targeting moiety for the biological marker is less than 250 nM. Preferably, the binding affinity of the targeting moiety for the biological marker is 5 nM, 10 nM, 40 nM, 90 nM, 130 nM or 170 nM.

Preferably, the length of the first polynucleotide or the length of the second polynucleotide are 4 nt to 500 nt. Preferably, the length of the first polynucleotide or the length of the second polynucleotide are 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 55, 60, 65, 70, 75, 80, 85, 90, 95, 100, 120, 160, 220, 300, 400, or 480 nt.

Preferably, the binding affinity between the first linker and the second linker is less than 250 nM. Preferably, the binding affinity between the first linker and the second linker is 2, 10, 25, 50, 62, 70, 85, 100, 102, 110, 125, 150, 162, 170, 185, 200, 202, 210, 225, or 250 nM.

Preferably, the first linker or the second linker is conjugated to a native functional group of the targeting unit or a surface of the cell, wherein the native functional group is an amino acid, a sugar, or an amine.

Preferably, the targeting moiety is a peptide, protein, or aptamer.

Preferably, the CD16 receptor is a CD16a receptor or a CD16b receptor.

Preferably, an expressed polynucleotide encoding the CD16 receptor is located on q arm of chromosome 1 at position 1q23.3.

Preferably, the cell is non-tumorigenic in an immune compromised mouse.

Preferably, after being irradiated with γ-ray, the cell is non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human natural killer cell further comprises an inactive tumor suppressor gene or a mutated and highly expressed oncogene.

Preferably, the human natural killer cell is capable of mediating an antibody-dependent cell cytotoxicity (ADCC) response, and the human natural killer cell is a male cell.

Preferably, the number of the human natural killer cells in the composition is at least 5×10⁵ and the human natural killer cells are in an amount equal to or more than 5% by number, based on the total number of the cells in the composition as 100%.

Preferably, the subject is a human.

Preferably, the method is for treating cancer selected form Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in feta, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembiyoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, other cancer, and combinations thereof.

Preferably, the biological marker is selected form carbohydrates, glycolipids, glycoproteins, CD (cluster of differentiation) antigens present on cells of a hematopoietic lineage such as CD2, CD4, CD8, CD21, etc.), γ-glutamyltranspeptidase; an adhesion protein (e.g., ICAM-1, ICAM-2, ELAM-1, VCAM-1); hormone, growth factor, cytokine, and other ligand receptors; ion channels; and the membrane-bound form of an immunoglobulin μ. Chain.

Preferably, the CD (cluster of differentiation) antigens present on cells of a hematopoietic lineage is CD2, CD4, CD8, CD21, or other CD (cluster of differentiation) antigens.

Preferably, the adhesion protein is ICAM-1, ICAM-2, ELAM-1, VCAM-1, or other adhesion protein.

Preferably, the cell further comprising a synthetic, genetically modified and/or deliberately delivered polynucleotide encoding chimeric antigen receptor (CAR) comprising a target-binding single-chain variable fragment (scFv) against an antigen selected from CD2, CD3 delta, CD3 epsilon, CD3 gamma, CD4, CD7, CD8a, CD8, CD1 1a (ITGAL), CD1 1b (ITGAM), CD1 1c (ITGAX), CD1 1d (ITGAD), CD 18 (ITGB2), CD 19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD1 1a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2, BCMA, PD-L1, and combinations thereof.

Preferably, the cell is capable of mediating an antibody-dependent cell cytotoxicity (ADCC) response.

The present invention provides a method of treating cancer, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of a human natural killer cell to a subject in need thereof; the human natural killer cell comprises a synthetic, genetically modified and/or deliberately delivered polynucleotide encoding chimeric antigen receptor (CAR) comprising a target-binding single-chain variable fragment (scFv) against an antigen selected from CD2, CD3 delta, CD3 epsilon, CD3 gamma, CD4, CD7, CD8a, CD8, CD1 1a (ITGAL), CD1 1b (ITGAM), CD1 1c (ITGAX), CD1 1d (ITGAD), CD 18 (ITGB2), CD 19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD1 1a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2, BCMA, PD-L1, and combinations thereof, and the human natural killer cell is (A) deposited at NPMD having the deposit number NITE BP-03017; or (B) having the following characteristics:

i) expressing a CD16 receptor,

ii) retaining its capability to proliferate after subculture for at least 3 months, and

iii) x) not including synthetic, genetically modified and/or deliberately delivered polynucleotide encoding the CD16 receptor, or y) by using ddPCR system to analyze the genomic DNA of the human natural killer cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

Preferably, the number of the human natural killer cells in the composition is at least 5×10⁵ and the human natural killer cells are in an amount equal to or more than 5% by number, based on the total number of the cells in the composition as 100%.

Preferably, the subject is a human.

Preferably, the method is for treating cancer selected form Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in feta, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembiyoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma. Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, other cancer, and combinations thereof.

The term “oNK” refers to (a) the isolated non-transgenic human CD16⁺ natural killer cell line derived from the population of human peripheral blood natural killer cells having the deposit number ATCC CRL-2407; (b) the non-transgenic human CD16⁺ natural killer cell line obtained by culturing the cell of (a) for multiple days with the culture method disclosed in the embodiments 2.1; (c) the cell which is deposited at NPMID having the deposit number NITE BP-030117; or (d) a human natural killer cell having the following characteristics:

i) expressing a CD16 receptor;

ii) retaining its capability to proliferate after subculture for at least 3 months; and

x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, Wherein the sequence of the CD16 F176F probe is SEQ ID NO: 11 and the sequence of the CD16 F176V probe is SEQ ID NO: 12

### BRIEF DESCRIPTION OF THE DRAW

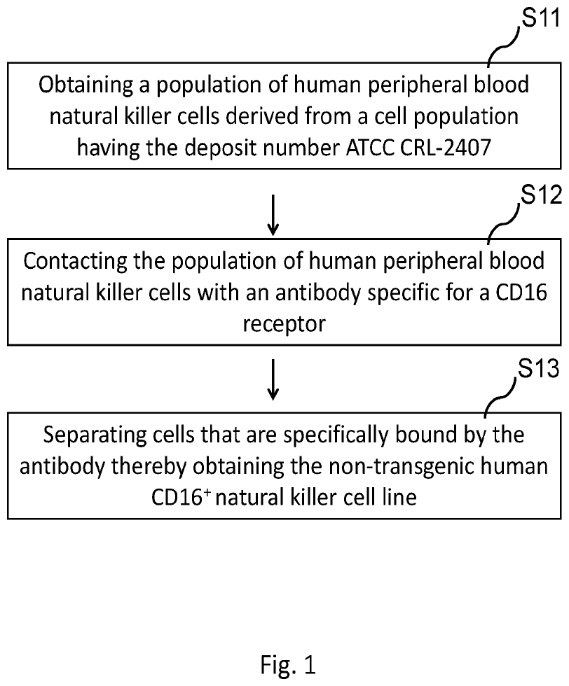

FIG. 1 is the flowchart of obtaining a non-transgenic human CD16⁺ natural killer cell line.

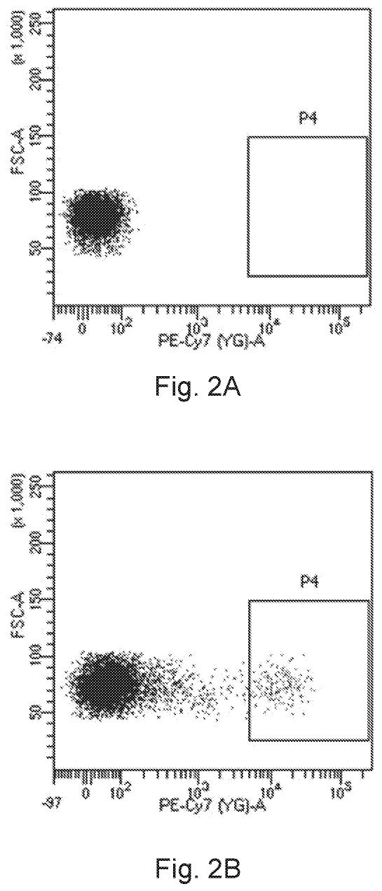

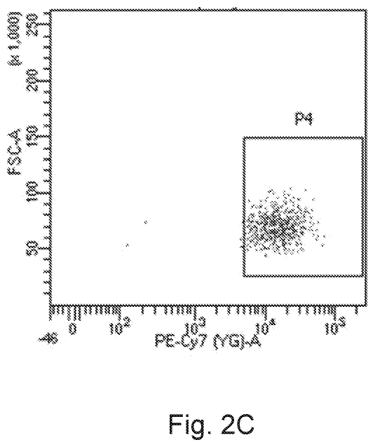

FIG. 2A is the two-dimensional dot plot presenting the population of human peripheral blood natural killer cells without the labeling of CD16 fluorescent labeled antibody, wherein the population of human peripheral blood natural killer cells is derived from the cell population having the deposit number ATCC CRL-2407.

FIG. 2B is the two-dimensional dot plot presenting the population of human peripheral blood natural killer cells with the labeling of CD16 fluorescent labeled antibody, wherein the population of human peripheral blood natural killer cells is derived from the cell population having the deposit number ATCC CRL-2407.

FIG. 2C is the two-dimensional dot plot presenting CD16 receptor expressing cell isolated from the population of human peripheral blood natural killer cells by the labeling of CD16 fluorescent labeled antibody.

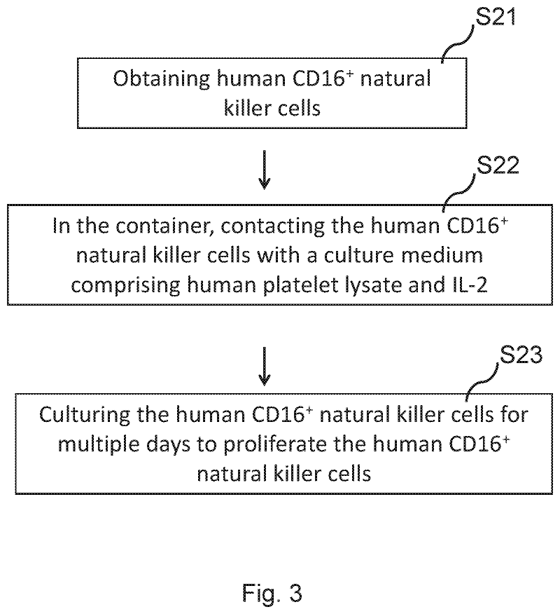

FIG. 3 is the flowchart of culturing human CD16⁺ natural killer cells.

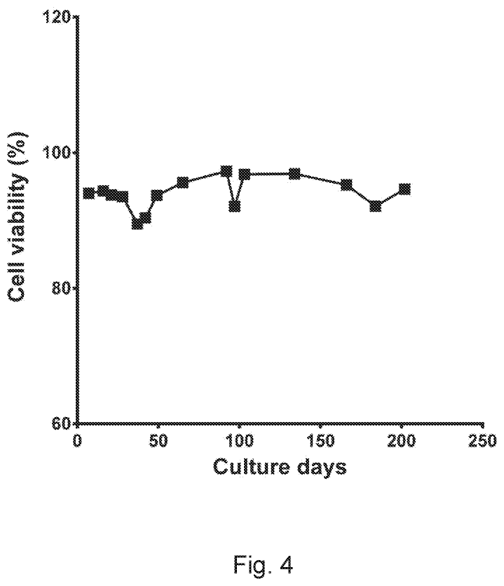

FIG. 4 is the line graph presenting the cell viability of non-transgenic human CD16⁺ natural killer cell line after different days of culturing.

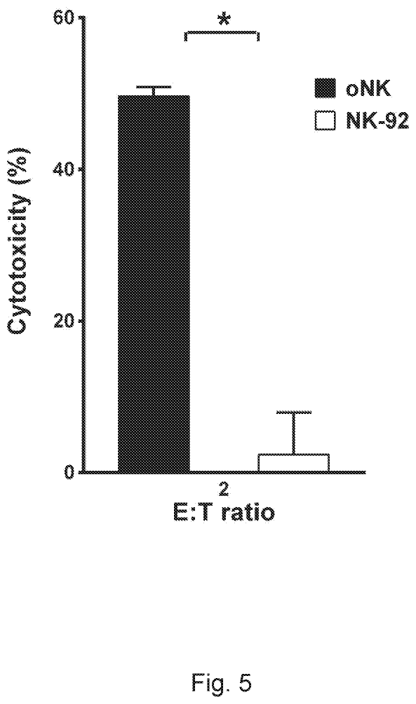

FIG. 5 is the bar chart presenting the comparison of the cytotoxic function between the cultured non-transgenic human CD16⁺ natural killer cell line and the NK-92 cell line to kill cancer cells.

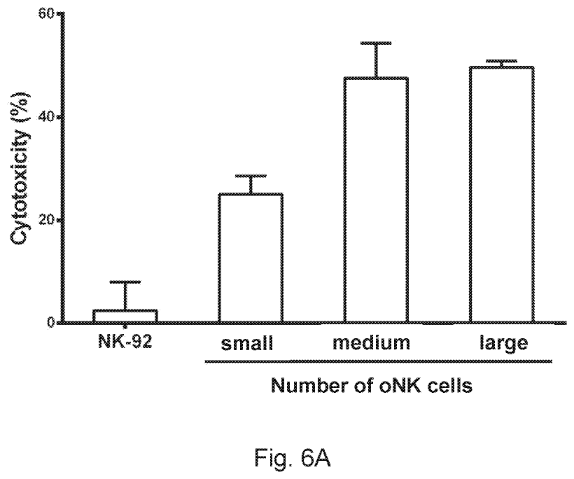

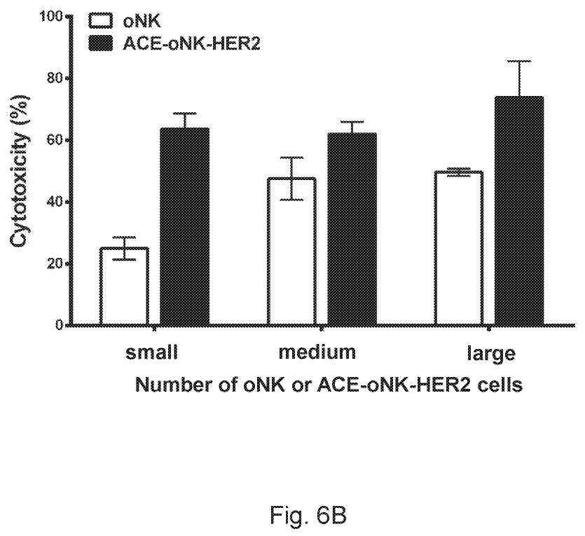

FIG. 6A is the bar chart presenting the comparison of the cytotoxic activity between different numbers of non-transgenic human CD16⁺ natural killer cell line to kill cancer cells.

FIG. 6B is the bar chart presenting the comparison of the cytotoxic activity between different numbers of anti-HER2 antibody-conjugated non-transgenic human CD16⁺ natural killer cell line to kill cancer cells through ADCC process.

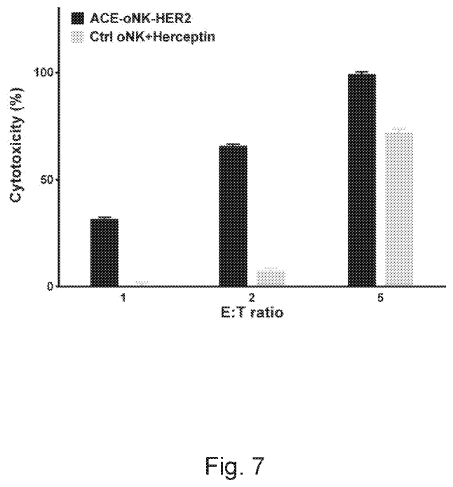

FIG. 7 is the bar chart presenting the comparison of the cytotoxic function between the anti-HER2 antibody-conjugated non-transgenic human CD16⁺ natural killer cell line and the anti-HER2 antibody co-cultured non-transgenic human CD16⁺ natural killer cell line to kill cancer cells through ADCC process.

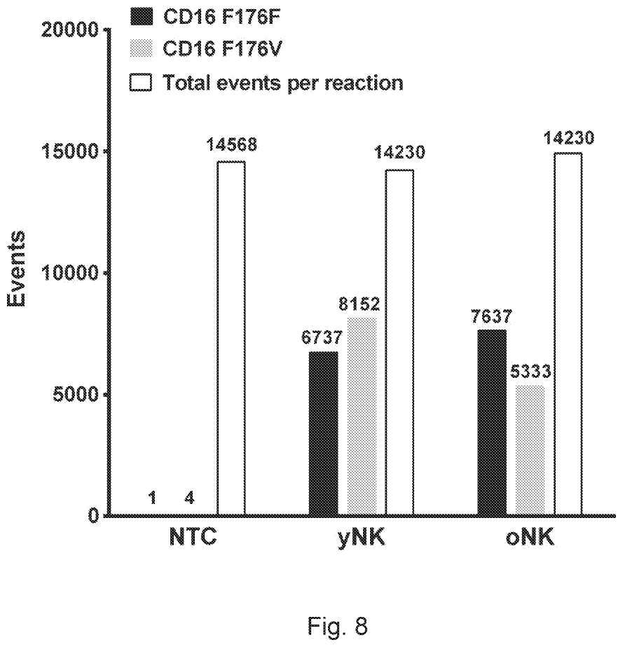

FIG. 8 is the bar chart presenting the comparison of genotype between the non-transgenic human CD16⁺ natural killer cell line and the CD16-transgenic NK-92 cell line.

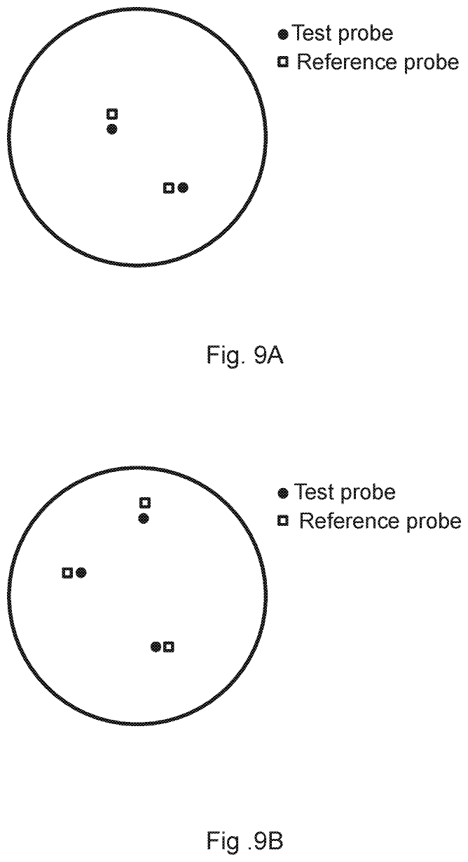

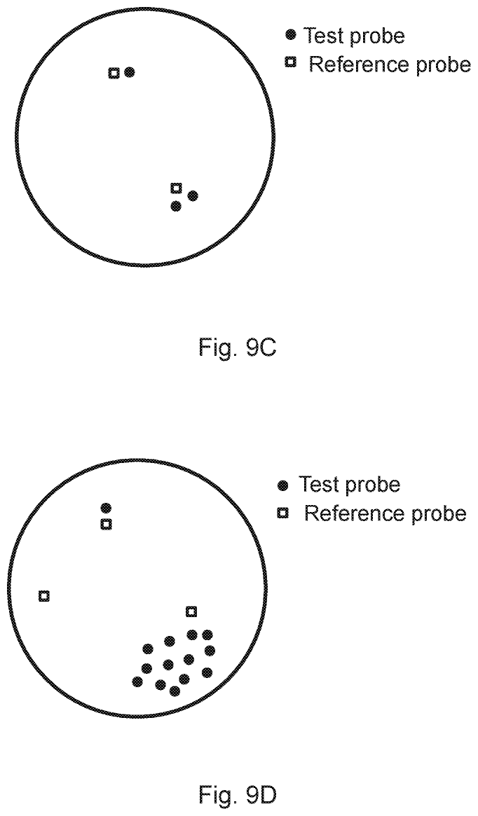

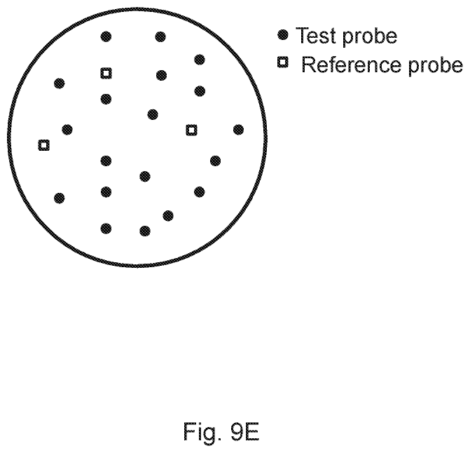

FIG. 9A-9E illustrates the principle by which two-color FISH analysis with a CD16a receptor gene-specific test probe labeled in one color and a reference probe labeled in another color can be applied to detect transgenic, synthetic, genetically modified, or deliberately delivered DNA sequence encoding the CD 16a receptor in human natural killer cells.

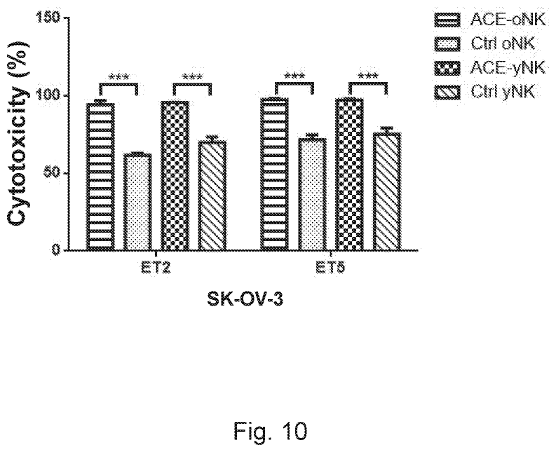

FIG. 10 is the bar chart presenting the cytotoxic function of non-transgenic human CD16⁺ natural killer cell line to kill cancer cells through ADCC process.

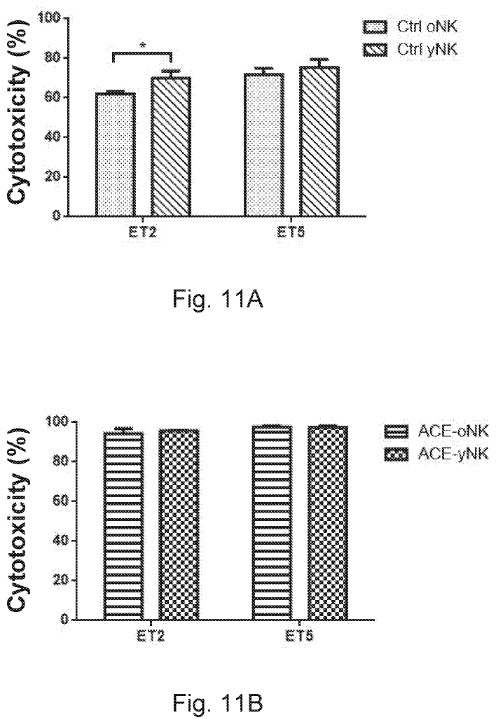

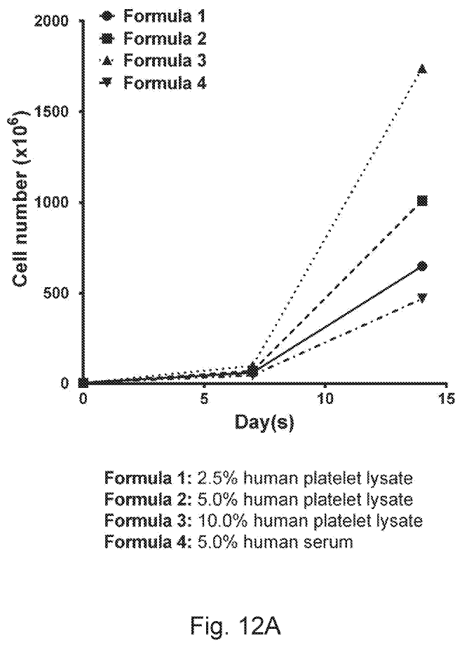

FIG. 11A is the bar chart presenting the comparison of the cytotoxic function between the non-transgenic human CD16⁺ natural killer cell line and the CD16-transgenic NK-92 cell line to kill cancer cells at different effector (E) to target (T) ratio.

FIG. 11B is the bar chart presenting the comparison of the cytotoxic function between the non-transgenic human CD16⁺ natural killer cell line and the CD16-transgenic NK-92 cell line to kill cancer cells through ADCC process at different effector (E) to target (T) ratio.

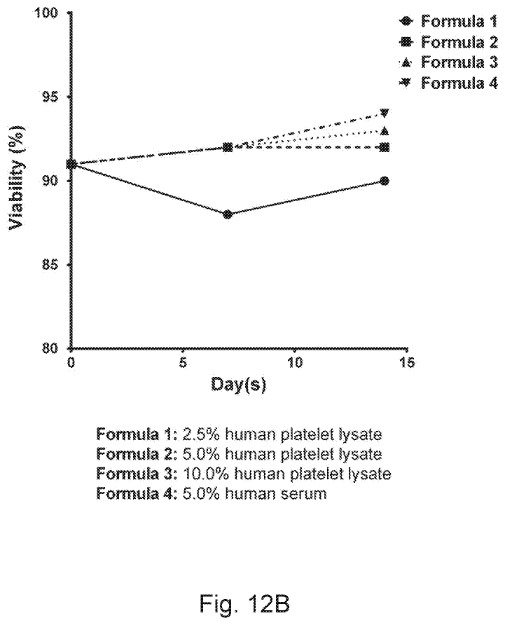

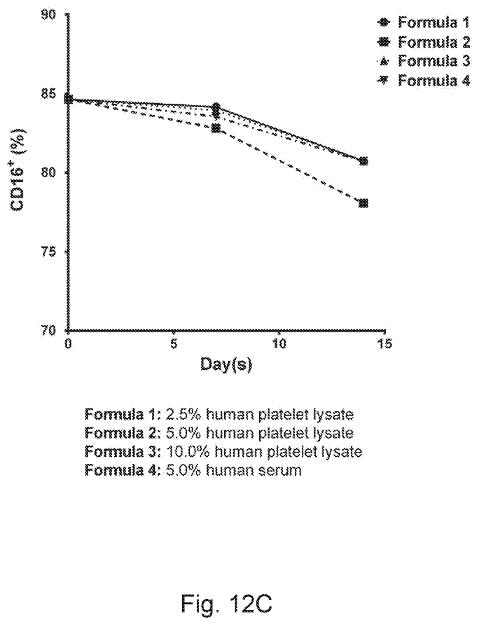

FIG. 12A is the line graph presenting the effect of human platelet lysate on total cell number after different days of culturing human CD16⁺ natural killer cell line.

FIG. 12B is the line graph presenting the effect of human platelet lysate on cell viability after different days of culturing human CD16⁺ natural killer cell line.

FIG. 12C is the line graph presenting the effect of human platelet lysate on maintaining the expression of CD16 after different days of culturing human CD16⁺ natural killer cell line.

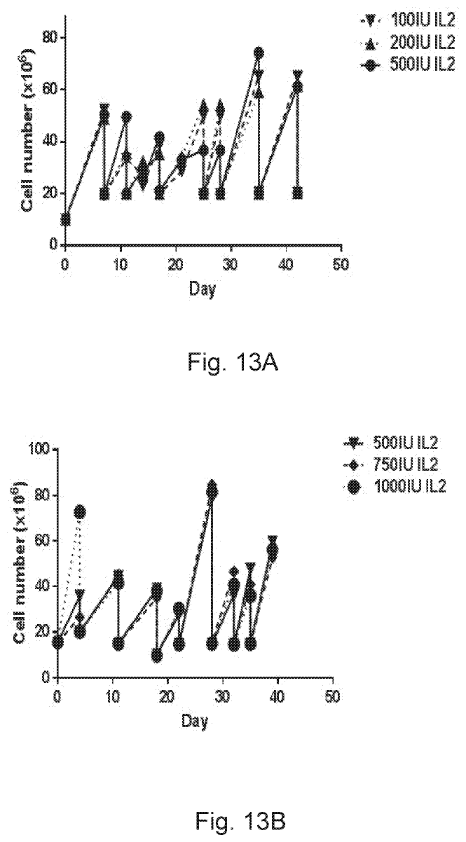

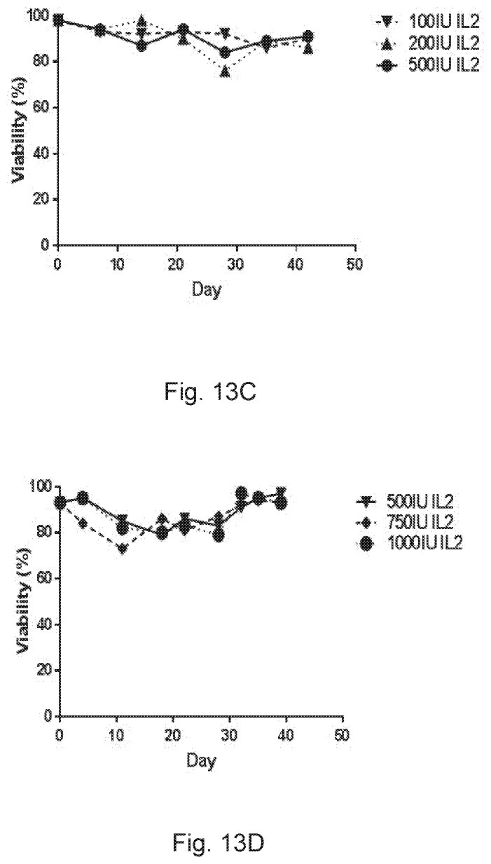

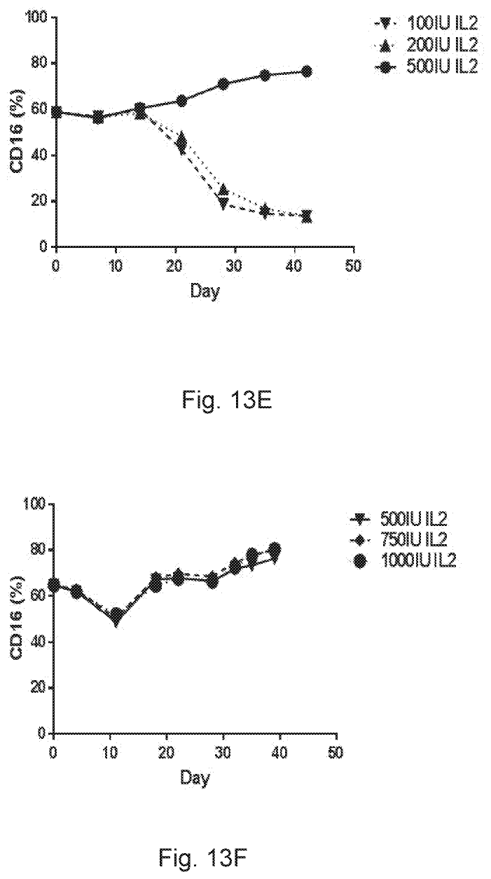

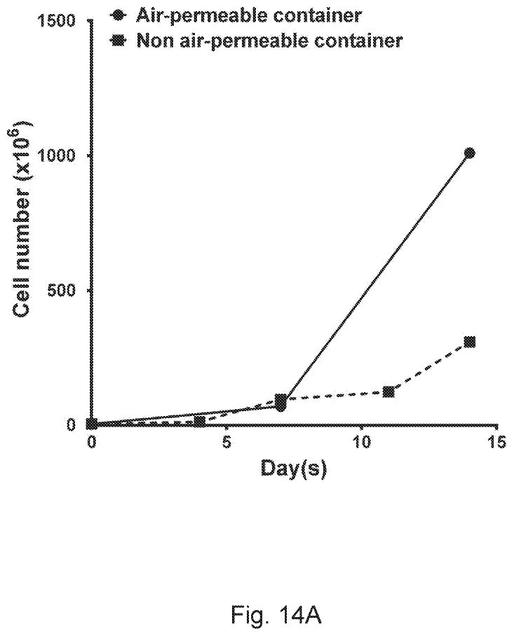

FIG. 13A is the line graph presenting the effect of low concentration of IL-2 on total cell number after different days of culturing human CD16⁺ natural killer cell line.

FIG. 13B is the line graph presenting the effect of high concentration of IL-2 on total cell number after different days of culturing human CD16⁺ natural killer cell line.

FIG. 13C is the line graph presenting the effect of low concentration of IL-2 on cell viability after different days of culturing human CD16⁺ natural killer cell line.

FIG. 13D is the line graph presenting the effect of high concentration of IL-2 on cell viability after different days of culturing human CD16⁺ natural killer cell line,

FIG. 13E is the line graph presenting the effect of low concentration of IL-2 on maintaining the expression of CD16 after different days of culturing human CD16⁺ natural killer cell line.

FIG. 13F is the line graph presenting the effect of high concentration of IL-2 on maintaining the expression of CD16 after different days of culturing human CD16⁺ natural killer cell line.

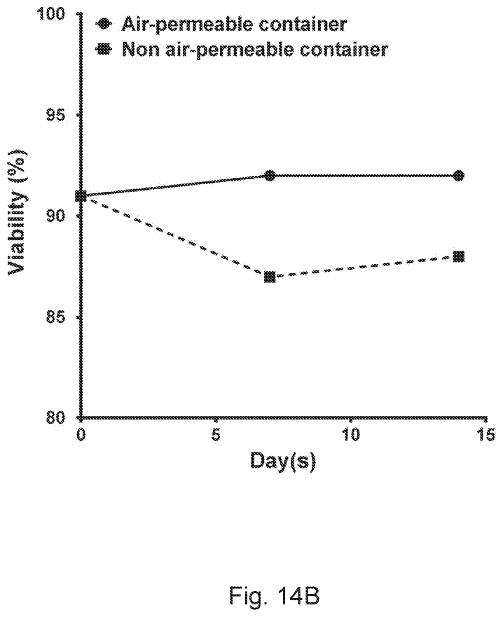

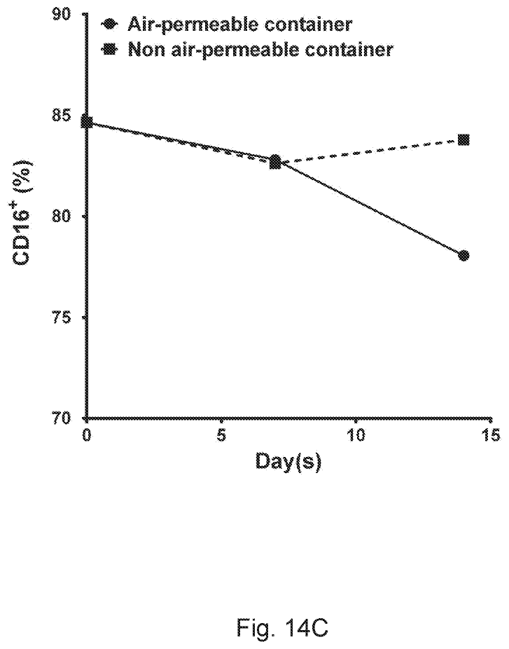

FIG. 14A is the line graph presenting the effect of air-permeable container on total cell number after different days of culturing human CD16⁺ natural killer cell line.

FIG. 14B is the line graph presenting the effect of air-permeable container on cell viability after different days of culturing human CD16⁺ natural killer cell line.

FIG. 14C is the line graph presenting the effect of air-permeable container on maintaining the expression of CD16 after different days of culturing human CD16⁺ natural killer cell line.

### DETAILED DESCRIPTION OF THE INVENTION

The following is a detailed description using the embodiments of the present invention as well as the techniques and features of the present invention, however, these embodiments are not intended to limit the invention, any changes and modifications made without departing from the spirit and scope of the invention by anyone who is familiar with this technology are intended to be included in the scope of the invention.

### Embodiment 1: Obtaining Non-Transgenic Human CD16⁺ Natural Killer Cell Line

Please refer to FIG. 1. FIG. 1 is the flowchart of obtaining a non-transgenic human CD16⁺ natural killer cell line. The method for obtaining a non-transgenic human CD16⁺ natural killer cell line in the present invention comprises at least the following steps:

Step S11: Obtaining a population of human peripheral blood natural killer cells derived from a cell population having the deposit number ATCC CRL-2407; Step S12: Contacting the population of human peripheral blood natural killer cells with an antibody specific for a CD16 receptor; Step S13: Separating cells that are specifically bound by the antibody thereby obtaining the non-transgenic human CD16⁺ natural killer cell line

Preferably, in Step S12, the CD16 receptor is a CD16a receptor.

Preferably, flow cytometry, bead, or any material with antibody-modified surface is used to separate the cells that are specifically bound by the antibody in Step S13.

Preferably, the term “non-transgenic human CD16 natural killer cell line” refers to non-genetically modified human CD16⁺ natural killer cell line and/or human CD16⁺ natural killer cell line without synthetic or exogenous polynucleotide sequence.

Detailed description of preferred embodiment is elaborated below.

### Embodiment 1.1 Label and Sorting of Non-Transgenic Human CD16⁺ Natural Killer Cell Line

This embodiment consists of an experimental group and a control group. The population of human peripheral blood natural killer cells with the deposit number ATCC CRL-2407 was centrifuged at a speed of 100˜1000×g for 3˜5 minutes. The supernatant was removed, and the population of human peripheral blood natural killer cells was resuspended with a buffer. The population of human peripheral blood natural killer cells was evenly distributed into the cell culture dishes of the control group and the experimental group. The population of human peripheral blood natural killer cells of experimental group was cultured in said cell culture dishes and then mixed with CD16 fluorescently labeled antibody (CD16-PE-Cy7, an antibody against CD16a receptor and CD16b receptor) to label the cells expressing CD16 receptor in the population of human peripheral blood natural killer cells; while the population of human peripheral blood natural killer cells of the control group was mixed with an equal volume of the buffer. The cells in the experimental group and control group were separately centrifuged, the supernatant was removed, and a sorting buffer was added to adjust the cell concentration to 1×10⁷ cells per mL. Finally, the cell population of the experimental and control groups were analyzed using a cell sorter.

Wherein, the buffer was Pre-Sort buffer. Flow cytometry sample preparation buffers, or Dulbecco's phosphate buffer saline (DPBS). The sorting buffer was Pre-Sort buffer, Flow cytometry sample preparation buffers, or Dulbecco's phosphate buffer saline (DPBS) supplemented with fetal bovine serum (FBS). The cell sorter was, for example, a flow cytometer of Becton Dickinson-FACSAria Illu model.

Preferably, the sorting buffer comprises 0.1˜10% (Volume percent, vol %, v/v) Fetal bovine serum (Fetal Bovine Serum, FBS).

Preferably, the sorting time is 1 hour, and the sorting speed is 50-70000 events/second.

After using the forward scatter (FSC) and side scatter (SSC) of the cell sorter to analyze 110,000 particles in the control group and the experimental p respectively, 6771 particles in the 10,000 particle in the control group were cells (that the number of cells is 67.7% when the number of particles is 100%), and 6944 particles in the 10,000 particles in the experimental group were cells (when the number of particles is 100″, the number of cells accounted for 69.4%).

The results for fluorescent analysis of the control group cells are shown in FIG. 2A, FIG. 2A is the two-dimensional dot plot presenting the population of human peripheral blood natural killer cells without the labeling of CD16 fluorescent labeled antibody, wherein the population of human peripheral blood natural killer cells is derived from the cell population having the deposit number ATCC CRL-2407; The result for fluorescent analysis of experimental group cells are shown in FIG. 2B, FIG. 2B is the two-dimensional dot plot presenting the population of human peripheral blood natural killer cells with the labeling of CD16 fluorescent labeled antibody, wherein the population of human peripheral blood natural blood natural killer cells is derived from the cell population having the deposit number ATCC CRL-2407.

In FIG. 2A and FIG. 2E, the abscissa is the relative value of PE-Cy7 fluorescence intensity (the CD16 fluorescent labeled antibody used in this experiment emits PE-Cy7 fluorescence), and the ordinate is the relative value of forward scatter (FSC) intensity.

The results in FIG. 2A show that all or the 6771 cells analyzed in the control group did 2.5 not emit PE-Cy7 fluorescence (0 cell in the rectangular region). Thus, in the absence of CD16-PE-cy7 fluorescent labeled antibody labeling, there were no other radiated light with similar wavelengths to PE-Cy7 fluorescent dye interfering the experimental result of control group cells.

The results in FIG. 2B show that most of the 6944 cells analyzed in the experimental group did not have PE-Cy7 fluorescence, and only a few cells had PE-Cy7 fluorescence (there are only 174 cells in the rectangular area). Thus, it is known that 6944 of 10,000 particles in the experimental group are cells of which 174 cells exhibit CD16 receptor, which means only 1.7% of the particles are cells expressing CD16 receptor (174÷10000=1.7%), and only 2.5%˜2.6% of the cells are cells expressing CD16 receptor (174÷6944 2.6%). In the experimental group, based on the condition of the cell concentration is 1×10⁷ cells per mL, each mL of cell solution in experimental group contained roughly 2.6×10⁵ cells expressing the CD16 receptor.

Cells expressing the CD16 receptor were sorted from the experimental group cells in order to obtain high-purity CD16⁺ cells (hereinafter referred to as “purified CD16⁺ cell population”, “isolated oNK”, or “isolated non-transgenic human CD16⁺ natural killer cell line”.)

Please refer to FIG. 2C, FIG. 2C is the two-dimensional dot plot presenting CD16 receptor expressing cell isolated from the population of human peripheral blood natural killer cells by the labeling of CD16 fluorescent labeled antibody. The results in FIG. 2C are shown that most cells in the purified CD16⁺ cell population emit PE-Cy7 fluorescence, and the purity of the cells expressing CD16 receptor is as high as 99%.

The aforesaid cells expressing CD16 receptor in the purified CD16⁺ cell population are non-transgenic cells; all of the aforesaid cells expressing CD16 receptor in the purified CD16⁺ cell population have the feature of CD3⁻CD56⁺ after analysis, they can be continuously subcultured and are non-tumorigenic; therefore, aforesaid cell expressing CD16 receptor in the purified CD16⁺ cell population is a novel non-transgenic human CD16⁺ natural killer cell line.

### Embodiment 2: Culturing Human CD16⁺ Natural Killer Cells

Please refer to FIG. 3. FIG. 3 is the flowchart of culturing human CD16⁺ natural killer cells. The method for culturing human CD16⁺ natural killer cells comprises at least the following steps:

Step S21: Obtaining human CD16⁺ natural killer cells;

Step S22: In the container, contacting the human CD16⁺ natural killer cells with a culture medium comprising human platelet lysate and IL-2; and

Step S23: Culturing the human CD16⁺ natural killer cells for multiple days to proliferate the human CD16⁺ natural killer cells.

The following describes a specific embodiment of culturing a non-transgenic human CD16⁺ natural killer cell line by the present invention, but the application of the invention is not limited thereto, which means the invention can also be used for culturing other human CD16⁺ natural killer cells. For example, primary CD16⁺ natural killer cell isolated from autologous or allogeneic blood, CD16-transgenic NK-92 cell line, or other human CD16⁺ natural killer cells.

### Embodiment 2.1 Culturing Non-Transgenic Human CD16⁺ Natural Killer Cell Line

Step S21′: The purified CD16⁺ cell population (the proportion of cell expressing CD16 receptor was as high as 99%) sorted by Embodiment 1 was centrifuged and the supernatant was removed.

Step S22′: after resuspending the cells with 1 mL of cell culture medium, the cell suspension was placed in a first container to make the first container contain 6 54×10⁵ non-transgenic human CD16⁺ natural killer cell line in 40 mL cell culture medium; the cell culture medium comprises: 0.5%-30% (Volume percent, vol %, v/v) Human platelet lysate; 100-3000 IU/mL Interleukin 2 (IL-2); and DMEM culture medium (Dulbecco's Modified Eagle Medium), alpha modification of Eagle's minimum essential medium, or XVIVO 10 culture medium.

Step S23′: After multiple days of culture, a composition substantially enriched in human CD16⁺ natural killer cells was obtained, and in the composition substantially enriched in human CD16⁺ natural killer cells, the number of non-transgenic human CD16⁺ natural killer cell line is at lease 5×10⁵; the multiple days are, for example, 1 days to 3 years.

Preferably, the multiple days are 1 week, 2 weeks, 3 weeks, 1 month, 2 months, 3 months, 4 months, 5 months, 6 months, 7 months, 8 months, 9 months, 10 months, 11 months, 1 year, 2 years, or 3 years.

Preferably, the cell culture medium comprises 0.5%, 1%, 1.5%, 1.6%, 2%, 2.5%, 2.6%, 3%, 3.5%, 3.6%, 4%, 4.5%, 4.6%, 5.0%, 5.1%, 5.5%, 5.6%, 6%, 6.1%, 6.5%, 6.6%, 7%, 7.1%, 7.5%, 7.6%, 8%, 8.1%, 8.5%, 8.6%, 9%, 9.1%, 9.5%, 9.6%, or 10% (Volume percent, vol %, v/v) human platelet lysate.

Preferably, the cell culture medium comprises 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 IU/mL IL-2.

Preferably, Step S23 ‘further comprise substeps:

Step S231′: after multiple days of culture, the number of the cells in the cell culture medium reached the first cell number, and the first cell number was 1.25×10⁶˜5×10⁶;

Step S232′: The cell suspension was placed in a second container to make the number of cells in the second container be the first cell number; after multiple days of culture, the number of the cells reached the second cell number, and the second cell number was 5×10⁷˜1×10⁹; and

Step S233′: The cell suspension was placed in a third container to make the number of cells in the third container be the second cell number; after multiple days of culture, the number of the cells reached the third cell number in order to obtain a composition substantially enriched in human CD16⁺ natural killer cells; the third cell number was, for example, 5×10⁹, or 1×10⁴⁰.

Wherein, the first container was, for example, a T25 cell culture flask (T25 flask), or G-Rex 6-well cell culture plate. The second and third containers comprised a gas permeable but water impermeable membrane, or, the second and third container can make concentration of the dissolved oxygen fully aerated or make the concentration of the dissolved glucose in the culture medium maintain in the 1500-5000 mg/L. Preferably, the second container was, for example, G-Rex 100M bottle (Product number 81100, WILSON WOLF, USA), the third container was, for example, G-Rex-500M (Product number 85500S, WILSON WOLF, USA). Please refer to the product manual of these containers for the instruction of using G-Rex 6M 6-well cell culture plate-, G-Rex 100M bottle, and G-Rex-500M.

In the steps S23′ and S231′˜S233′, 0.5%-30% (Volume percent, vol %, v/v) Human platelet lysate and 100-3000 IU/mL Interleukin 2 (IL-2) were added to a medium for culturing the cells And the medium is for example, DMEM culture medium (Dulbecco's Modified Eagle Medium), alpha modification of Eagle's minimum essential medium, XVIVO 10 culture medium, or X-VIVO 10 Serum-free Hemapoietic Cell Medium.

In the steps S23′ and S231′˜S233′, the cells were incubated under the condition of 37° C. and 5% carbon dioxide.

### Embodiment 2.2 Detecting Cell Viability of the Cultured Cells Obtained from Embodiment 2.1

Each sample of the cell suspensions, which were obtained by culturing for different days with the culture method disclosed in the embodiments 2.1, was mixed with an equal volume of Trypan blue, and the number of cells and the cell survival rate were observed.

The experimental results showed that after culturing for 7, 16, 21, 28, 37, 42, 49, 65, 92, 97, 103, 134, 166, 184, and 202 days, the number of cells respectively reached 1.61×10⁶, 1.01×10⁹, 2.53×10⁹, 5.06×10⁹, 1.01×10¹⁰, 1.62×10¹⁰, 3.24×10¹⁰, 1.13×10¹¹, 1.81×10¹⁵, 3.25×10¹⁶, 6.50×10¹⁷, 1.35×10²², 3.24×10²⁷, 1.30×10³³, and 1.04×10³⁹. Please refer to FIG. 4. FIG. 4 shows that cell viability was maintained at 84-97% after 7, 16, 21, 28, 37, 42, 49, 65, 92, 97, 103, 134, 166, 184, and 202 days of culture of non-transgenic human CD16⁺ natural killer cell line. Thus, culturing the non-transgenic human CD16⁺ natural killer cell line with the culture method of the present invention can make the cells number expand at lease 1.59×10³³ folds (1.04×10³⁹)÷(6.54×10⁵)≈1.59×10³³), while effectively maintaining the cell viability rate after the proliferation.

### Embodiment 3: Detection of Cell Condition and Cell Surface Markers

### Embodiment 3.1 Long-Term Culture of Non-Transgenic Human CD16⁺ Natural Killer Cell Line by the Culture Method of the Present Invention

There are two experimental trials in this embodiment. The first batch of the purified CD16⁺ cell population and the second batch of the purified CD16⁺ cell population (the proportions of cells expressing the CD16 receptor in both of the batches were as high as 99%) were sorted by the method of Embodiment 1.1, then the first batch of the purified CD16⁺ cell population and the second batch of the purified CD16⁺ cell population were cultured respectively by the culture method of Embodiment 2.1 to obtain the cell suspensions of the first experimental trial and the cell suspensions of the second experimental trial. The first batch of the purified CD16⁺ cell population was cultured for 35 days in total, while the second batch of the purified CD16⁺ cell population was cultured for at least a long period of time until day 202.

### Embodiment 3.2 Detecting the Condition of the Cultured Cells

Each sample of the cell suspensions, which were obtained at different time points in Embodiment 3.1, was centrifuged; the supernatant was removed, the cells were resuspended in the buffer, then mix with 1 μL of propidium iodide (PI). The cell sorter or flow cytometer was used to detect whether the cells were stained with propidium iodide to determine the percentage of cells that were undergoing apoptosis or have died.

### Embodiment 3.3 Detection of CD56, CD3, and CD2 Surface Markers of the Cultured Cells

Each sample of the cell suspensions, which were obtained at different time points in Embodiment 3.1, was centrifuged; the supernatant was removed, the cells were resuspended in the buffer, then mixed with 1 μL of CD56 fluorescent labeled antibody (Cat. No. 318304, Biolegend, USA), 1 μL of CD3 fluorescent labeled antibody (Cat. No. 300410, Biolegend, USA), and 1 μL of CD2 fluorescent labeled antibody (Cat. No. 300222, Biolegend, USA) to simultaneously label cells expressing CD56 molecule, CD3 molecule, and/or CD2 molecule. Finally, the cell sorter or flow cytometer was used to analyze whether the cells exhibited CD56 molecules, CD3 molecules, and/or CD2 molecules, and the percentage of cells with various cell surface makers was calculated.

### Embodiment 3.4 Detection of CD16 Surface Markers of the Cultured Cells

Each sample of the cell suspensions, which were obtained at different time points in Embodiment 3.1, was centrifuged; the supernatant was removed, the cells were resuspended in the buffer, then mixed with 1 μL of CD16 fluorescent labeled antibody (Cat. No. 302016, Biolegend, USA) to label cells expressing the CD16 receptor. Finally, the cell sorter was used to analyze whether the cells exhibited CD16 receptor, and the percentage of cells with CD16 receptor was calculated.

### Embodiment 3.5 Detection of Cytotoxic Function of the Cultured Cells

xCELLigence Real Time Cell Analysis System (xCELLigence RTCA system, ACEA Biosciences Inc., USA) was used in this embodiment to detect the cytotoxic ability of the cultured cell toward target cells. This embodiment comprised a 96 well xCELLigence E-Plate to carry out cytotoxicity test, and the wells in xCELLigence E-Plate were divided into control wells, experimental wells, and target cell maximum lysis control well. The effector cells used in this embodiment were the cell suspensions obtained by culturing at different time points in Embodiment 3.1, and the target cells were SK-OV-3 cell line (HTB-77, purchased from ATCC), which is an adherent ovarian cancer cell line. SK-OV-3 cells were seeded in control well, experimental well, and target cell maximum lysis control well, so that each well-contained 20000 SK-OV-3 cells, and then allowed it to sit 30 minutes.

A sample of the cell suspension obtained in Embodiment 3.1 was added to the experimental well, and the ratio of the number of effector cell to the number of SK-OV-3 cells (target cells) was 2, 5 and 10; added a tenth equal volume of lysis buffer to the sample of cell suspension into target cell maximum lysis control well; any sample or lysis buffer was not added to control well. The xCELLigence E-Plate was placed in the xCELLigence Real Time Cell Analysis System to detect real time change in the cell index (CI) under the condition of 37° C. and 5% carbon dioxide.

Wherein, the greater the number of target cells attached to the bottom of the xCELLigence E-Plate, the higher the cell index detected by the xCELLigence Real Time Cell Analysis System. Therefore, the cell index can be used to convert the percentage of target cells that are lysed in the experimental well. The formula used to convert the cell index to the percentage of target cells that are lysed in the experimental well is:

Percentage of lysed target cell (%)=1−[(cell index of experimental well−cell index of target cell maximum lysis control well)(cell index of control well-cell index of target cell maximum lysis control well)]×100%

Please refer to Table 1 and Table 2. Table 1 shows the results of the cell suspensions obtained from the first experimental trial, and Table 2 shows the results of the cell suspension obtained from the second experimental trial.

In Table 1, the first column “day” indicates the number of culture days; the second column “PI⁺” indicates the percentage of cells undergoing apoptosis or have died, based on the total number of the cells in the cell suspension as 100%; since natural killer cells, CD4⁺ T cells, and CD8⁺ T cells all exhibit CD56⁺ (Pernick, N, 2018), so the third column “CD56⁺” indicates the percentage of the total number of natural killer cells, CD4⁺ T cells, and CD8⁺ T cells, based on the total number of the cells in the cell suspension as 100%; since T cells all exhibit CD3⁺ (Pernick, N, 2018), the fourth column “CD3⁺” indicates the percentage of cells that are not T cells, based on the total number of the cells in the cell suspension as 100%; since natural killer cells, peripheral blood T cells, and most thymocytes all exhibit CD2⁺ (Pernick, N, 2018) and the cells to be bested in Embodiment 3 are derived from peripheral blood, so the fifth column “CD2⁺” indicates the percentage of the total number of natural killer cells and T cells, based on the total number of the cells in the cell suspension as 100%; the sixth column “CD56⁺CD3⁺” indicates the percentage of natural killer cells, based on the total number of the cells in the cell suspension as 100%; the seventh column “CD56⁺CD2⁺” indicates percentage of the total number of natural killer cells and T cells, based on the total number of the cells in the cell suspension as 100%; since natural killer cell and macrophage exhibit CD16⁺ (Pernick, N, 2018), and CD16 is involved in Antibody-dependent cell cytotoxicity (ADCC), the eighth column “CD16⁺” indicates the percentage of the total number of natural killer cells and macrophages with ADCC function, based on the total number of the cells in the cell suspension as 100%; the ninth column “CD56⁺CD16⁺” indicates the percentage of natural killer cells with ADCC function, based on the total number of natural killer cells (i.e., CD56⁺CD3⁻ cells) as 100%.

The indication of the first to eighth columns in Table 2 is the same as in Table 1; when the ninth column “killing test” marks “✓” symbol, this indicates that the cytotoxic function of the cells in the cell suspension at certain time point was simultaneously tested and confirmed that the cells have cytotoxic function.

Table 1 shows that (1) in the cell suspension that was obtained after the first batch of the purified CD16⁺ cell population (wherein the proportion of human CD16⁺ natural killer cell line is as high as 99%) was cultured for 7˜35 days, the percentage of cells undergoing apoptosis or have died is 5.65%˜7.34%, thus, the percentage of cell survival during culture is 92.66%˜94.35%; (2) in the cell suspension that was obtained after the first batch of the purified CD16⁺ cell population was cultured for 7˜35 days, the percentage of total number of natural killer cell, CD4⁺ T cell, and CD8⁺ T cell is 99.0˜99.56%, based on the total number of the cells in the cell suspension as 100%; (3) in the cell suspension that was obtained after the first batch of the purified CD16⁺ cell population was cultured for 7˜35 days, the percentage of cells that are not T cells is 99.8˜100%, based on the total number of the cells in the cell suspension as 100%; (4) in the cell suspension that was obtained after the first batch of the purified CD16⁺ cell population was cultured for 7˜35 days, the percentage of total number of natural killer cell and T cell is 98.0˜99.22%, based on the total number of the cells in the cell suspension as 100%; (5) in the cell suspension that was obtained after the first batch of the purified CD16⁺ cell population was cultured for 7˜35 days, the percentage of natural killer cells is 98.21˜98.76%, based on the total number of the cells in the cell suspension as 100%; (6) in the cell suspension that was obtained after the first batch of the purified CD16⁺ cell population was cultured for 7˜35 days, the percentage of total number of natural killer cell and T cell is 98.7˜99.33%, based on the total number of the cells in the cell suspension as 100%; (7) in the cell suspension that was obtained after the first batch of the purified CD16⁺ cell population was cultured for 7˜35 days, the percentage of the total number of natural killer cells and macrophages with ADCC function is 90.17˜92.36%, based on the total number of the cells in the cell suspension as 100%; (8) in the cell suspension that was obtained after the first batch of the purified CD16″ cell population was cultured for 7˜35 days, the percentage of natural killer cell with ADCC function is 88.79˜92.11%, based on the total number of natural killer cell (i.e., CD56⁺CD3⁻ cell) as 100%.

Table 1 the test result of cell condition and cell surface marker of the cell suspension obtained by culturing the first batch of the purified CD16⁺ cell population.

| | PI⁺ | CD56⁺ | CD3⁻ | CD2⁺ | CD56⁺CD3⁻ | CD56⁺CD2⁺ | CD16⁺ | CD56⁺/CD16⁺ |
|---|---|---|---|---|---|---|---|---|
| | (% of total | (% of total | (% of total | (% of total | (% of total | (% of total | (% of total | (% of CD56⁺CD3⁻ |
| Day | cells) | cells) | cells) | cells) | cells) | cells) | cells) | cells) |
| 7 | 6.54 | 99.45 | 100.00 | 98.08 | 98.76 | 99.01 | 90.17 | — |
| 16 | 5.65 | 99.08 | 99.96 | 98.86 | 98.21 | 98.78 | 90.35 | — |
| 21 | 7.34 | 99.56 | 99.9 | 98.75 | 98.71 | 99.33 | 90.67 | — |
| 23 | — | — | — | — | — | — | — | 88.79 |
| 26 | — | — | — | — | — | — | — | 90.51 |
| 28 | 7.18 | 99.33 | 99.88 | 99.22 | 98.29 | 99.15 | 92.36 | — |
| 30 | — | — | — | — | — | — | — | 92.11 |
| 35 | — | — | — | — | — | — | — | 91.37 |

Table 2 shows that (1) in the cell suspension that was obtained after the second batch of the purified CD16⁺ cell population (wherein the proportion of human CD16⁺ natural killer cell line is as high as 99%) was cultured for 7-202 days, the percentage of cells undergoing apoptosis or have died is 2.7%-10.5%, thus, the percentage of cell survival during culture is 89.5%-97.3%; (2) in the cell suspension that was obtained after the second batch of the purified CD16⁺ cell population was cultured for 7-202 days, the percentage of total number of natural killer cell, CD4⁺ T cell, and CD8⁺ T cell is 98.85%-99.65%, based on the total number of the cells in the cell suspension as 100%; (3) in the cell suspension that was obtained after the second batch of the purified CD16⁺ cell population was cultured for 7-202 days, the percentage of cells that are not T cells is 99.82%-100%, based on the total number of the cells in the cell suspension as 100%; (4) in the cell suspension that was obtained after the second batch of the purified CD16⁺ cell population was cultured for 7-202 clays, the percentage of total number of natural killer cell and T cell is 94.5%-99.68%, based on the total number of the cells in the cell suspension as 100%; (5) in the cell suspension that was obtained after the second batch of the purified CD16⁺ cell population was cultured for 7-202 days, the percentage of natural killer cells is 97.65%-99.05%, based on the total number of the cells in the cell suspension as 100%; (6) in the cell suspension that was obtained after the second batch of the purified CD16⁺ cell population was cultured for 7-202 days, the percentage of total number of natural killer cell and T cell is 97.83%-99.61%, based on the total number of the cells in the cell suspension as 100%; (7) in the cell suspension that was obtained after the second batch of the purified CD16⁺ cell population was cultured for 7-202 days, the percentage of the total number of natural killer cells and macrophages with ADCC function is 83.88%-94.04%%, based on the total number of the cells in the cell suspension as 100%; (8) The cell in the cell suspension that was obtained after the second batch of the purified CD16⁺ cell population was cultured for 7-202 days was confirmed to have cytotoxic function.

The cell suspension obtained by culturing for 28 days with the culture method disclosed in the embodiment 2.1 has been deposited at NPMD with the deposit number NITE BP-03017. The results disclosed in this invention indicate that the oNK cell line could retain its capability to proliferate after subculture for at least 3 months and thus may comprised deregulated genes responsible for cell growth control (e.g. the oNK cell line may comprised an inactive tumor suppressor gene, or a mutated and highly expressed oncogene).

Table 2 the test results of cell condition, cell surface marker and cytotoxicity of the cell suspension obtained by culturing the second batch of the purified CD16⁺ cell population.

| | | PI⁺ | CD56⁺ | CD3⁻ | CD2⁺ | CD56⁺CD3⁻ | CD56⁺CD2⁺ | CD16⁺ | |
|---|---|---|---|---|---|---|---|---|---|
| | Total cell | (% of total | (% of total | (% of total | (% of total | (% of total | (% of total | (% of total | Killing |
| Day | number | cells) | cells) | cells) | cells) | cells) | cells) | cells) | test |
| 7 | 1.61 × 10⁶ | 5.97 | 99.45 | 100.00 | 98.08 | 98.76 | 99.01 | 90.17 | |
| 16 | 1.01 × 10⁹ | 5.65 | 99.09 | 99.96 | 98.86 | 98.21 | 98.8 | 90.36 | |
| 21 | 2.53 × 10⁹ | 6.2 | 99.56 | 99.91 | 98.75 | 98.72 | 99.33 | 90.7 | |
| 28 | 5.06 × 10⁹ | 6.46 | 99.33 | 99.88 | 99.22 | 98.29 | 99.15 | 92.36 | |
| 37 | 1.01 × 10¹⁰ | 10.5 | 98.85 | 99.99 | 98.48 | 97.65 | 98.66 | 91.96 | |
| 42 | 1.62 × 10¹⁰ | 9.63 | 99.15 | 100.00 | 98.24 | 98.06 | 98.8 | 93.09 | |
| 49 | 3.24 × 10¹⁰ | 6.31 | 98.99 | 100.00 | 94.5 | 97.71 | 97.83 | 94.04 | |
| 65 | 1.13 × 10¹¹ | 4.41 | 99.15 | 99.99 | 98.55 | 97.81 | 98.85 | 90.35 | |
| 92 | 1.81 × 10¹⁵ | 2.7 | 99.62 | 99.99 | 99.43 | 98.58 | 99.42 | 85.99 | ✓ |
| 97 | 3.25 × 10¹⁶ | 7.91 | 99.23 | 99.90 | 99.58 | 98.3 | 99.05 | 86.98 | ✓ |
| 103 | 6.54 × 10¹⁷ | 3.17 | 99.65 | 99.82 | 99.5 | 98.71 | 99.45 | 83.88 | ✓ |
| 134 | 1.35 × 10²² | 3.09 | 99.62 | 99.99 | 99.68 | 98.75 | 99.42 | 86.18 | ✓ |
| 166 | 3.24 × 10²⁷ | 4.74 | 99.17 | 100.00 | 99.06 | 99.05 | 99.61 | 89.93 | ✓ |
| 184 | 1.30 × 10³³ | 7.87 | 99.61 | 99.99 | 98.23 | 98.77 | 99.37 | 92.38 | ✓ |
| 202 | 1.04 × 10³⁹ | 5.36 | 99.59 | 99.96 | 97.52 | 98.94 | 99.33 | 93.02 | ✓ |

### Embodiment 4: Non-Tumorigenicity of Non-Transgenic Human CD16⁺ Natural Killer Cell Line

Six to eight-week-old female BALB/c nude mice (purchased from The Jackson Laboratory or BioLasco, Taiwan) were used in this Embodiment. 30 mice were randomly assigned into six groups, which were a SK-OV-3 group, Raji group, Daudi group, oNK group. γ-ray irradiated ACE-oNK group, and DPBS group.

A human ovarian cancer cell line “SK-OV-3” (Purchased from ATCC; The deposit number is ATCC HTB-77), human B lymphoblastoid cell lines “Raji” (Purchased from ATCC; The deposit number is ATCC CCL-86) and “Daudi” (Purchased from ATCC; The deposit number is ATCC CCL-213), a cell suspension that was obtained by culturing for 88 days with the culture method disclosed in the embodiments 2.1 (88-day cultured oNK suspension of the present invention, refer to as 88-day cultured oNK suspension), and a γ-ray irradiated ACE-oNK cell suspension were used in this Embodiment. The method for preparing γ-ray irradiated ACE-oNK cell suspension was described below.

γ-ray irradiated ACE-oNK cell suspension: the cell suspension that was obtained by culturing for 84 days with the culture method disclosed in the embodiments 2.1 (84-day cultured oNK suspension of the present invention, refer to as 84-day cultured oNK suspension) were gamma irradiated at dose 10 Gy. After binding Trastuzumab to cells in the γ-ray irradiated 84-day cultured oNK suspension using a cell linker and a Trastuzumab linker which are complementary, they-ray irradiated ACE-oNK cell suspension were obtained.

The procedure of binding Trastuzumab to cells (e.g., natural killer cells, cells in the 60-day cultured oNK suspension, cells in the γ-ray irradiated 60-day cultured oNK suspension) was as follows: (A) The step of preparing cell linker and binding the cell linker to the cell in order to prepare a cell-ssDNA conjugate; (B) The step of preparing Trastuzumab linker and binding the Trastuzumab linker to Trastuzumab in order to prepare the Trastuzumab-ssDNA conjugate; (C) Mixing cell-ssDNA conjugate and Trastuzumab-ssDNA conjugate to combine cell-ssDNA conjugate and Trastuzumab-ssDNA conjugate through the cell linker and its complementary sequence on the Trastuzumab linker in order to prepare Trastuzumab-conjugated cells.

Wherein the step (A) of preparing cell linker and binding the cell linker to the cell comprises the following steps (a1)˜(a4):

Step (a1) A first single strand DNA was obtained, wherein the sequence of the first single strand DNA was SEQ ID NO:5, SEQ ID NO:6, or SEQ ID NO:7.

Step (a2) The 5′ end of the first single strand DNA was modified as 5′ end thiol-modified first single strand DNA to obtain the cell linker stock. The cell linker stock is also commercially available from Integrated DNA Technologies. Actual methods of modification are known, or will be apparent, to those skilled in the art (Zimmermann, J, 2010).

Step (a3) 10-500 μL cell linker stock and 0.1-10 μL NHS-Maleimide (commercially available from Fisher Scientific) were mixed and incubated for 1-60 minute(s).

Step (a4) The mixture obtained from Step (a3) were mixed with 1×10⁶-1×10⁸ cells and incubated for 1-60 minutes to obtain cell-ssDNA conjugate.

The step (B) of preparing Trastuzumab linker and binding the Trastuzumab linker to Trastuzumab comprises the following steps (b1)˜(b4):

Step (b1) A second single strand DNA was obtained, wherein the sequence of the second single strand DNA was SEQ ID NO:8, SEQ ID NO:9, or SEQ ID NO:10, and the sequence of the second single strand DNA is the complementary strand to the first single strand DNA.

Step (b2) The 5′ end of the second single strand DNA was modified as 5′ end thiol-modified second single strand DNA to obtain a Trastuzumab linker stock. The Trastuzumab linker stock is also commercially available from Integrated DNA Technologies. Actual methods of modification are known, or will be apparent, to those skilled in the art (Zimmermann, J, 2010).

Step (b3) 10-500 μL Trastuzumab linker stock and 0.1-10 μL NHS-Maleimide (commercially available from Fisher Scientific) were mixed and incubated for 1-60 minute(s).

Step (b4) The mixture obtained from Step (b3) were mixed with 10-100 μL Trastuzumab stock (commercially available from Roche) and incubated for 10 minutes to 3 hours to obtain Trastuzumab-ssDNA conjugate.

The cell-ssDNA conjugate and the Trastuzumab-ssDNA conjugate were mixed to obtain Trastuzumab-conjugated cell such as cells in the γ-ray irradiated ACE-oNK cell suspension.

1×10⁷ SK-OV-3 cells, 1×10⁷ Raji cells, 1×10⁷ Daudi cells, 1×10⁷ cells in the 60-day cultured oNK suspension, and 1×10⁷ cells in γ-ray irradiated ACE-oNK cell suspension were suspended respectively in 100 μL of Dulbecco's Phosphate-Buffered Saline (DPBS) to obtain different cell suspensions. The cell suspensions and 100 μL of DPBS were subcutaneously implanted in female BALB/c nude mice in SK-OV-3 group, Raji group, Daudi group, oNK group, γ-ray irradiated ACE-oNK group, and DPBS group on Day 0 respectively. Tumor growth in each mouse was observed on Day 14, Day 21, Day 24, Day 42, and Day 59, and the mice were euthanized on Day 59.

Please refer to Table 3. Table 3 shows the results of tumor formation in nude mice xenografted with different cell lines.

Table 3 shows that there was no tumor formation in the mice of DPBS groups (negative control group) throughout the study period (0/5, 0%), while all five mice in SK-OV-3 group (positive control group) developed tumors (5/5, 100%). For mice xenografted with lymphoma cell line Daudi, 4 out of 5 mice in Daudi group developed tumors (4/5, 80%) that lasted until end of study (Day 59). For mice xenografted with lymphoma cell line Raji, 1 out of 5 (1/5) mice harbored detectable tumor before Day 42, but then returned to unmeasurable size by end of study.

For mice xenografted with oNK cells or γ-ray irradiated ACE-oNK cells of the present invention, there was no tumor formation in mice in oNK group and γ-ray irradiated ACE-oNK group throughout the study period (0/5, 0%). These study results provide evidence that non-irradiated oNK cells and the Trastuzumab-conjugated irradiated ACE-oNK cells are non-tumorigenic and safe for future clinical application and disease treatment.

Table 3 the results of tumor formation in nude mice xenografted with different cell lines.

| | Tumor incidence | | | | |
|---|---|---|---|---|---|
| Cell type | Day 14 | Day 21 | Day 24 | Day 42 | Day 59 |
| SK-OV-3 | 5/5 | 5/5 | 5/5 | 5/5 | 5/5 |
| suspension | | | | | |
| Raji | 1/5 | 1/5 | 1/5 | 0/5 | 0/5 |
| suspension | | | | | |
| Daudi | 0/5 | 3/5 | 4/5 | 4/5 | 4/5 |
| suspension | | | | | |
| oNK | 0/5 | 0/5 | 0/5 | 0/5 | 0/5 |
| suspension | | | | | |
| ACE-oNK-HER2 | 0/5 | 0/5 | 0/5 | 0/5 | 0/5 |
| suspension | | | | | |
| DPBS | 0/5 | 0/5 | 0/5 | 0/5 | 0/5 |

### Embodiment 5: The Comparison of the Cytotoxic Activity Between the Cultured Non-Transgenic Human CD16⁺ Natural Killer Cell Line and NK-92 Cell Line

The experimental method of this embodiment is almost the same as that of Embodiment 3.5, except that (1) the effector cell used in this embodiment is \textcircled{1} the cell suspension that was obtained by culturing for 33 days with the culture method disclosed in the embodiments 2.1 (33-day cultured oNK suspension of the present invention, refer to as 33-day cultured oNK suspension wherein the proportion of human CD16⁺ natural killer cell line is 91.74%), or \textcircled{2} the population of human peripheral blood natural killer cells having the deposit number ATCC CRL-2407 (refer to as NK-92 suspension wherein the proportion of NK-92 cell line is at least 98% as shown in FIG. 2B and the NK-92 cell line is a CD16⁻ natural killer cell line); and (2) the ratio of the number of effector cells (the total cells in the 33-day cultured oNK suspension or the total cells in the NK-92 suspension) to the number of SK-OV-3 cells (target cells) is 2:1 (ET2).

Please refer to FIG. 5. FIG. 5 is the bar chart presenting the comparison of the cytotoxic function between the cultured non-transgenic human CD16⁺ natural killer cell line and NK-92 cell line to kill cancer cells. FIG. 5 shows that NK-92 cell line (a CD16⁻ natural killer cell line and thus unable to destroy cancer cells through ADCC process) only killed 2.40±5.52% of cancer cells, whereas oNK cells (non-transgenic human CD16⁺ natural killer cells that were not linked to or co-cultured with IgG antibodies targeting the tumor-associated antigens and thus not activated to induce ADCC reaction) killed 49.68±1.19% of cancer cells.

Thus, the result shows that: as compare with NK-92 cells (NK-92 is a CD16⁻ natural killer cell line and thus unable to destroy cancer cells through ADCC process), oNK cells that were not activated to induce ADCC reaction could cause about 21-fold increase of cytotoxicity (49.68÷2.4=21). This is an unexpected result.

Moreover, based on this result, applicant believe that after isolating human CD16⁺ natural killer cell line from the 33-day cultured oNK suspension (cultured oNK) and isolating CD16⁻ natural killer cell line (NK-92) from the NK-92 suspension, similar unexpected result could be observed.

### Embodiment 6: The Comparison of the Cytotoxic Activity Between Different Amount of Non-Transgenic Human CD16⁺ Natural Killer Cell Line

The experimental method of this embodiment is almost the same as that of Embodiment 3.5, except that (1) the effector cell used in this embodiment is \textcircled{1} the cell suspension that was obtained by culturing for X days with the culture method disclosed in the embodiments 2.1 (X-day cultured oNK suspension of the present invention wherein the proportion of human CD16⁺ natural killer cell line is 8.91%, refer to as suspension with small number of oNK cells), \textcircled{2} the cell suspension that was obtained by culturing for Y days with the culture method disclosed in the embodiments 2.1 Y-day cultured oNK of the present invention wherein the proportion of human CD16⁺ natural killer cell line is 64.15%, refer to as suspension with medium number of oNK cells), \textcircled{3} the cell suspension that was obtained by culturing for Z days with the culture method disclosed in the embodiments 2.1 (Z-day cultured oNK of the present invention wherein the proportion of human CD16⁺ natural killer cell line is 91.74%, refer to as suspension with large number of oNK cells), \textcircled{4} the population of human peripheral blood natural killer cells with the deposit number ATCC CRL-2407 (refer to as NK-92 suspension wherein the proportion of NK-92 cell line is at least 98% as shown in FIG. 2B and the NK-92 cell line is a CD16⁻ natural killer cell line), \textcircled{5} suspension with small number of ACE-oNK-HER2 cells, © suspension with medium number of ACE-oNK-HER2 cells, or \textcircled{7} suspension with large number of ACE-oNK-HER2 cells; and (2) the ratio of the number of effector cells (the total cells in the suspension with small number of oNK cells, in the suspension with medium number of oNK cells, in the suspension with large number of oNK cells, in the NK-92 suspension, in the suspension with small number of ACE-oNK-HER2 cells, in the suspension with medium number of ACE-oNK-HER2 cells, or in the suspension with large number of ACE-oNK-HER2 cells) to the number of SKOV-3 cells (target cells) is 2:1 (ET2).

The method for preparing the suspension with small number of ACE-oNK-HER2 cells, the suspension with medium number of ACE-oNK-HER2 cells, and the suspension with large number of ACE-oNK-HER2 cells were described below.

The suspension with small number of ACE-oNK-HER2 cells: the total cells in “the suspension with small number of oNK cells” were linked with Trastuzumab by using a cell linker and a Trastuzumab linker that are complementary, and therefore the suspension with small number of ACE-oNK-HER2 cells were obtained wherein the proportion of ACE-oNK-HER2 cells is about 8.91%.

The suspension with medium number of ACE-oNK-HER2 cells: the total cells in “the suspension with medium number of oNK cells” were linked with Trastuzumab by using a cell linker and a Trastuzumab linker that are complementary, and therefore the suspension with medium number of ACE-oNK-HER2 cells was obtained wherein the proportion of ACE-oNK-HER2 cells is about 64.15%.

The suspension with large number of ACE-oNK-HER2 cells: the total cells in “the suspension with large number of oNK cells” were linked with Trastuzumab by using a cell linker and a Trastuzumab linker that are complementary, and therefore the suspension with large number of ACE-oNK-HER2 cells was obtained wherein the proportion of ACE-oNK-HER2 cells is about 91.74%.

The procedure of binding Trastuzumab to cells (cells in the suspension with small number of oNK cells, the suspension with medium number of oNK cells, or the suspension with large number of oNK cells) is same as that of Embodiment 4.

Please refer to FIG. 6A and FIG. 6B. FIG. 6A is the bar chart presenting the comparison of the cytotoxic activity between different numbers of non-transgenic human CD16⁺ natural killer cell line to kill cancer cells. FIG. 6B is the bar chart presenting the comparison of the cytotoxic activity between different numbers of anti-HER2 antibody-conjugated non-transgenic human CD16⁺ natural killer cell line to kill cancer cells through ADCC process.

FIG. 6A shows that NK-92 cell line (a CD16⁻ natural killer cell line and thus unable to destroy cancer cells through ADCC process) only killed 2.40±5.52% of cancer cells; small number of oNK cells (non-transgenic human CD16⁺ natural killer cells that were not linked to or co-cultured with IgG antibodies targeting the tumor-associated antigens and thus not activated to induce ADCC reaction) killed 25.00±3.60% of cancer cells; medium number of oNK cells (non-transgenic human CD16⁺ natural killer cells that were not linked to or co-cultured with IgG antibodies targeting the tumor-associated antigens and thus not activated to induce ADCC reaction) killed 47.60±6.80% of cancer cells; large number of oNK cells (non-transgenic human CD16⁺ natural killer cells that were not linked to or co-cultured with IgG antibodies targeting the tumor-associated antigens and thus not activated to induce ADCC reaction) killed 49.68±1.19% of cancer cells.

Thus, the result shows that: as compare with NK-92 cells (NK-92 is a CD16⁻ natural killer cell line and thus unable to destroy cancer cells through ADCC process), the suspension with small number of oNK cells (wherein the proportion of human CD16⁺ natural killer cell line is 8.91%) is enough to cause about 10-fold increase of cytotoxicity (25÷2.4=10). This is an unexpected result. Therefore, it indicated that human CD16⁺ natural killer cell line in an amount equal to or more than 5% by number is enough to kill cancer cells, based on the total number of the cells in the composition as 100%. Based on this result, applicant believes that similar unexpected result could be observed in clinical trials.

The result also shows that the suspension with medium or large number of oNK cells (wherein the proportion of human CD16⁺ natural killer cell line is 64.15% or 91.74%) could cause about 20-21 fold increase of cytotoxicity (47.60÷2.4=20; 49.68÷2.4=21). Therefore, it indicates that the more the human CD16⁺ natural killer cell line, the more the cancer cells are killed and then reach a plateau as the human CD16⁺ natural killer cell line in an amount equal to about 60%-65% by number, based on the total number of the cells in the composition as 100%. Based on this result, applicant believes that similar result could be observed in clinical trials.

FIG. 6B shows that small number of oNK cells killed 25.00±3.60% of cancer cells; medium number of oNK cells killed 47.60±6.80% of cancer cells; large number of oNK cells killed 49.68±1.19% of cancer cells; small number of ACE-oNK-HER2 cells killed 63.70±5.00% of cancer cells; medium number of ACE-oNK-HER2 cells killed 62.00±4.00% of cancer cells; large number of ACE-oNK-HER2 cells killed 73.9±11.80% of cancer cells.

Thus, the result shows that: when the non-transgenic human CD16⁺ natural killer cell line obtained by the culture of the present invention was linked with antibodies targeting the tumor-associated antigens (such as Trastuzumab) by using a cell linker and a antibody liner (such as Trastuzumab linker) which are complementary and thus could be activated to induce ADCC reaction, the cytotoxic effect was significantly increased by 14.4%-38.7% (62.00%−47.60%=14.4%; 63.70%−25.00%=38.7%).

The result also shows that exogeneous targeting unit complexed-oNK cell (such as anti-HER2 antibody-conjugated oNK cells) in an amount equal to or more than 5% by number is enough to kill cancer cells through ADCC process; it also indicates that the more the exogeneous targeting unit complexed-oNK cell, the more the cancer cells are killed through ADCC process and reach a first plateau as the exogeneous targeting unit complexed-oNK cell in an amount equal to about 5%-10% by number, based on the total number of the cells in the composition as 100%. Based on this result, applicant believes that similar result could be observed in clinical trials.

### Embodiment 7: The Comparison of the Cytotoxic Activity Between the Anti-HER2 Antibody-Conjugated Non-Transgenic Human CD16⁺ Natural Killer Cell Line and the Anti-HER2 Antibody Co-Cultured Non-Transgenic Human CD16⁺ Natural Killer Cell Line

The experimental method of this embodiment is almost the same as that of Embodiment 3.5, except that (1) the effector cell used in this embodiment is \textcircled{1} cell suspensions obtained by culturing for 55 days with the culture method disclosed in the embodiments 2.1 (refer to as 55-day cultured oNK suspension), or \textcircled{2} cell suspension with ACE-oNK-HER2 cells (the total cells in “55-day cultured oNK suspension” were linked with Trastuzumab by using a cell linker and a Trastuzumab linker that are complementary as described in Embodiment 4); (2) the ratio of the number of effector cells (the total cells in the 55-day cultured oNK suspension or the total cells in the cell suspension with ACE-oNK-HER2 cells) to the number of SK-OV-3 cells (target cells) is 1:1 (ET1), 2:1 (ET2), or 5:1 (ET5); and (3) In the experimental wells for the 55-day cultured oNK suspension, equivalent amount of Trastuzumab as the total amount of the Trastuzumab linked to the cells in the cell suspension with ACE-oNK-HER2 cells at E:T ratio of 1 (0.55 ng), 2 (1.10 ng) and 5 (2.75 ng) was added. The detail procedure was described below.

The wells in xCELLigence E-Plate were divided into control wells, ACE-oNK-HER2 ET1 experimental well, ACE-oNK-HER2 ET2 experimental well, ACE-oNK-HER2 ET5 experimental well, oNK and Herceptin ET1 experimental well, oNK and Herceptin ET2 experimental well, oNK and Herceptin ET5 experimental well, and target cell maximum lysis control well. SK-OV-3 cells were seeded in control well, ACE-oNK-HER2 ET1 experimental well, ACE-oNK-HER2 ET2 experimental well, ACE-oNK-HER2 ET5 experimental well, oNK and Herceptin ET1 experimental well, oNK and Herceptin ET2 experimental well, oNK and Herceptin ET5 experimental well, and target cell maximum lysis control well, so that each well-contained 20000 SK-OV-3 cells, and then allowed it to sit 30 minutes.

20000, 40000, or 100000 cells in the cell suspension with ACE-oNK-HER2 cells was added to the ACE-oNK-HER2 ET1 experimental well, ACE-oNK-HER2 ET2 experimental well, and ACE-oNK-HER2 ET5 experimental well respectively; hence, the ratio of the number of effector cell (the total cells in the cell suspension with ACE-oNK-HER2 cells) to the number of SKOV-3 cells (target cells) was 1, 2 and 5.

Both of 20000, 40000, or 100000 cells in the 55-day cultured oNK suspension and 0.55, 1.10, or 2.75 ng of Trastuzumab (an antibody against HER2 protein with product name as Herceptin was purchased from Roche, Swiss) were added to the “oNK and Herceptin ET1 experimental well”, “oNK and Herceptin ET2 experimental well”, and “oNK and Herceptin ET5 experimental well” respectively. Therefore, the ratio of the number of effector cell (the total cells in the 55-day cultured oNK suspension) to the number of SK-OV-3 cells (target cells) was 1, 2 and 5; the amount of Trastuzumab in the “oNK and Herceptin ET1 experimental well”, “oNK and Herceptin ET2 experimental well”, or “oNK and Herceptin ET5 experimental well” was respectively same as the total amount of the Trastuzumab linked to the cells in the ACE-oNK-HER2 ET1 experimental well, ACE-oNK-HER2 ET2 experimental well, and ACE-oNK-HER2 ET5 experimental well.

Please refer to FIG. 7. FIG. 7 is the bar chart presenting the comparison of the cytotoxic function between the anti-HER2 antibody-conjugated non-transgenic human CD16⁺ natural killer cell line and the anti-HER2 antibody co-cultured non-transgenic human CD16⁺ natural killer cell line to kill cancer cells through ADCC process. FIG. 7 shows that oNK cells that were co-cultured with IgG antibodies targeting the tumor-associated antigens (and thus activated to induce ADCC reaction) only killed 0.00±2.10%, 7.30±1.40%, or 71.8±2.10% of cancer cells at E:T ratio of 1, 2, or 5 respectively, whereas ACE-oNK-HER2 cells that were linked (conjugated) with IgG antibodies targeting the tumor-associated antigens (and thus activated to induce ADCC reaction) killed 31.40±1.10%, 65.60±1.00%, or 99.10±1.30% of cancer cells at E:T ratio of 1, 2, or 5 respectively.

Thus, the result shows that: as compare with oNK cells that were co-cultured with IgG antibodies targeting the tumor-associated antigens, ACE-oNK-HER2 cells that were linked (conjugated) with IgG antibodies targeting the tumor-associated antigens could cause 9-∞ fold increase of cytotoxicity at lower doses (ET1 with 0.55 ng Trastuzumab, or ET2 with 1.10 ng Trastuzumab; 65.60÷7.30=9; 31.40÷0.00=∞; ∞ is a symbol that represents an infinitely large number). That is, “linking CD16⁺ natural killer cells with anti-tumor antigen antibody” (e.g. linking cultured oNK with Trastuzumab) makes an unexpected result, and linking CD16⁺ natural killer cells with anti-tumor antigen antibody” make effective and safer therapy based on lower dose treatment could be achieved.

Moreover, based on this result, applicant believes that after isolating human CD16⁺ natural killer cell line from the 55-day cultured oNK suspension (cultured oNK) and isolating Trastuzumab-linked CD16⁺ natural killer cells (ACE-oNK-HER2 cells) from the cell suspension with ACE-oNK-HER2 cells, similar unexpected result could be observed.

### Embodiment 8: Detection of Genomic DNA of Non-Transgenic Human CD16⁺ Natural Killer Cell Line

### Embodiment 8.1 Detection of DNA Sequence Encoding CD16 Receptor by Droplet Digital PCR (ddPCR

Droplet Digital PCR (ddPCR) was used in this embodiment to detect DNA sequence encoding CD16 receptor of cultured non-transgenic human CD16⁺ natural killer cell line in the present invention (oNK) or CD16-transgenic NK-92 cell line (yNK).

Cell suspensions obtained by culturing for M days with the culture method disclosed in the embodiments 2.1 (refer to as M-day cultured oNK suspension) and CD16-transgenic NK-92 cell line (Purchased from ATCC with the deposit number is ATCC PTA-6967; refer to as yNK) were used in this embodiment. Genomic DNA of yNK and cells in the M-day cultured oNK suspension were isolated by Blood & Cell Culture DNA Mini Kit (Purchased from Qiagen).

yNK sample or oNK sample: 50 ng genomic DNA isolated from yNK or M-day cultured oNK suspension was mixed with 10 μL ddPCR™ Supermix for Probes (2X) (Catalog number #1863026; Purchased from Bio-Rad), 1 μL BstXI restriction enzyme (Product name BstXI; Catalog number R0113S; Purchased form BioLabs), and 1 μL Mixture of CD16 F176F hydrolysis probe and CD16 F176V hydrolysis probe (Assay ID: C_25815666_10; Purchased form ThermoFisher; The Context Sequence [VIC/FAM]: TCTGAAGACACATTTTTACTCCCAA[C/A]AAGCCCCCTGCAGAAGTAGGAGCCG (SEQ ID NO: 41); https://www.thermofisher.com/order/genome-database/details/genotyping/C_25815666_10?CID=&ICID=&subtype=), and the final volume is 20 μL.

No-template control sample: water, 10 μL ddPCR™ Supermix for Probes (2X), BstXI restriction enzyme, and 1 μL Mixture of CD16 F176F hydrolysis probe and CD16 F176V hydrolysis probe were mixed, and the final volume is 20 μL.

ddPCR experiments were performed using the QX100/QX200 Droplet Digital PCR (ddPCR) system (Purchased from Bio-Rad). First, samples are placed into a QX100 or QX200 Droplet Generator (a machine in the QX100/QX200 Droplet Digital PCR system) to partition each sample into 15000-20000 droplets (nanoliter-sized droplet).

Second, the wells in the 96 well plate (Product name: DG8 cartridge; Purchased from Bio-Rad) were divided into no-template control well, yNK well, and oNK well, and these wells are for no-template control group (NTC group), yNK group, and oNK group respectively. Nanolized no-template control sample, yNK sample, and oNK sample were respectively transferred into the no-template control well, yNK well, and oNK well.

Third, for the PCR amplification process, thermocycling conditions were 95° C. for 10 min, 45 cycles of 95° C. for 15 s, and 60° C. for 1 min, followed by 98° C. for 10 min then hold at 4° C. The ramp rate for each step was set to 2° C./s.

CD16 F176F hydrolysis probe is a probe labeled with FAM reporter fluorophore, and CD16 F176V hydrolysis probe is a probe labeled with VIC reporter fluorophore. The main steps in the PCR amplification process are denaturation, annealing, and extension. During annealing, the hydrolysis probe (such as CD16 F176F hydrolysis probe or CD16 F176V hydrolysis probe) binds to the target sequence; then during extension, the reporter labeled at the 5′ end of the probe is cleaved and free reporter fluoresces. The sequence of CD16 F176F hydrolysis probe is SEQ ID NO:11 and thus is expected to be able to detect DNA sequence encoding CD16 receptor located on q arm of chromosome 1 at position 1q23.3; the sequence of CD16 F176V hydrolysis probe is SEQ ID NO:12 and is expected to be able to detect the synthetic DNA sequence in yNK.

Please note that the DNA sequence encoding CD16 receptor located on q arm of chromosome 1 at position 1q23.3 in oNK would be transcribed to CD16 F176F mRNA then translated to CD16 F176F protein, wherein the sequence of the DNA encoding CD16 receptor located on q arm of chromosome 1 at position 1q23.3 in oNK comprises SEQ ID NO:1, SEQ ID NO:2, or SEQ ID NO:19; the sequence of CD16 F176F mRNA comprises SEQ ID NO:13; the sequence of CD16 F176F protein comprises SEQ ID NO:3, SEQ ID NO:4, SEQ ID NO:14, or SEQ ID NO:20. The synthetic DNA sequence encoding CD16 receptor in yNK would be transcribed to CD16 F176V mRNA then translated to CD16 F176V protein, and the sequence of CD16 F176V mRNA is SEQ ID NO:15; the sequence of CD16 F176V protein is SEQ ID NO:16.

Forth, for droplet reading process, droplets were read using a QX100/QX200 Droplet Reader (a machine in the QX100/QX200 Droplet Digital PCR system), in which droplets were spaced out individually for fluorescence reading and therefore each droplet was analyzed individually using a two-color detection system (set to detect FAM and VIC). Positive droplets, which contain at least one copy of the target DNA molecule (such as CD16 F176F hydrolysis probe detected DNA molecule or CD16 F176V hydrolysis probe detected molecule), exhibit increased fluorescence compared to negative droplets.

Please refer to FIG. 8. FIG. 8 is the bar chart presenting the comparison of genotype between the non-transgenic human CD16⁺ natural killer cell line and the CD16-transgenic NK-92 cell line.

In NTC group, there were only 1 positive droplet containing CD16 F176F hydrolysis probe-detectable DNA molecule and 4 positive droplets containing CD16 F176V hydrolysis probe-detectable DNA molecules in total 14568 collected droplets (events). In yNK group, there were 6737 positive droplets containing CD16 F176F hydrolysis probe-detectable DNA molecules and 8152 positive droplets containing CD16 F176V hydrolysis probe-detectable DNA molecules in total 14230 collected droplets (events). In oNK group, there were 7637 positive droplets containing CD16 F176F hydrolysis probe-detectable DNA molecules and 5333 positive droplets containing CD16 F176V hydrolysis probe-detectable DNA molecule in total 14230 collected droplets (events).

Thus, the result shows that using ddPCR system to analyze the genomic DNA of yNK cells, the ratio of CD16 F176F hydrolysis probe-detectable DNA molecule to CD16 F176V hydrolysis probe-detectable DNA molecule was 0.83 (6737÷8152=0.83), whereas using ddPCR system to analyze the genomic DNA of oNK cells, the ratio of CD16 F176F hydrolysis probe-detectable DNA molecule to CD16 F176V hydrolysis probe-detectable DNA molecule was 1.43 (7637÷5333=1.43).

That is, by using ddPCR system to analyze the genomic DNA of human CD16⁺ natural killer cell line (oNK) in the present invention, the ratio of CD16 F176F hydrolysis probe-detectable DNA molecule to CD16 F176V hydrolysis probe-detectable DNA molecule was equal to or higher than 1 (the number of CD16 F176F probe detectable DNA molecule÷the number of CD16 F176V probe detectable DNA molecule ≥1).

Moreover, based on this result, applicant believes that after isolating human CD16⁺ natural killer cell line from the M-day cultured oNK suspension (cultured oNK), similar result could be observed.

Based on applicant's experience, another hydrolysis probes with sequence SEQ ID NO:17 or SEQ ID NO:18 could detect DNA sequence encoding CD16 receptor in other CD16-transgenic NK cells.

### Embodiment 8.2 Detection of DNA Sequence Encoding CD16 Receptor by Fluorescence In Situ Hybridization (FISH

Two-color fluorescence in situ hybridization (FISH) was used in this embodiment to detect transgenic, synthetic, genetically modified, or deliberately delivered DNA sequence encoding the CD 16a receptor in human natural killer cells.

The cultured non-transgenic human CD16⁺ natural killer cell line in the present invention (oNK) is used as an example to show the result of the human cell with no transgenic, synthetic, genetically modified, or deliberately delivered DNA sequence encoding the CD 16 receptor, whereas the CD16-transgenic NK-92 cell line (yNK) is used as an example to show the result of the human cell with transgenic, synthetic, genetically modified, or deliberately delivered DNA sequence encoding the CD 16 receptor.

For the detail, the isolated CD16⁺ NK cells (oNK cells) from the cell suspensions obtained by culturing for N days with the culture method disclosed in the embodiments 2.1 (refer to as N-day cultured oNK suspension) and CD16-transgenic NK-92 cell line (Purchased from ATCC with the deposit number is ATCC PTA-6967; refer to as yNK) were used in this embodiment.

Kallioniemi disclosed the details of the two-color fluorescence in situ hybridization (FISH) method in 1996, and a short extract is presented below.

First, nuclei from 1×10⁷ yNK cells or oNK cells (CD16⁺ NK cells) isolated form the N-day cultured oNK suspension are prepared according to protocols used in DNA flow cytometry (Kallioniemi et al., 1996; Vindelov et al., 1983). For the detail, the cell pellet is incubated in a hypotonic detergent solution and brief trypsin digestion.

Second, nuclei are dropped on microscope slides, air-dried, and fixed in methanol acetic acid.

Third, prior to hybridization, the target cells are treated with proteinase K or other proteolytic enzymes to improve probe penetration.

Forth, denaturation of target cells is usually accomplished by immersing slides in a denaturation solution (70% formamide, 2×SSC) for 2-4 min at 70° C., followed by ethanol fixation and dehydration. Denaturation time and temperature have to be optimized according to the characteristics of the target cells.

Fifth, Prior to hybridization, 20-60 ng of the first fluorescent dye-labeled FCGR3A FISH Probe (a test probe that could detect all of the human DNA sequence encoding CD16a receptor; Purchased from Empire Genomics), 20-60 ng of the second fluorescent dye-labeled Chromosome 1 Control Probe (a reference probe; Purchased from Empire Genomics), and blocking DNA (unlabeled Cot-1 or placental DNA) are added to a formamide-based hybridization buffer. It is necessary to use the blocking DNA when the probe contains repetitive sequences that will hybridize to multiple locations in the genome. Hybridization mixture is heated to 70° C. for 5 min to denature the probe fragments then applied on the target slide; a cover slip is applied and sealed with rubber cement. Hybridization is performed overnight at 37° C. in a moist chamber.

Sixth, unbound probes are washed.

Seventh, Target nuclei are counterstained with a DNA stain, typically propidium iodide or DAPI.

The hybridizations are evaluated with a regular high-quality epifluorescence microscope. Almost any recent microscope model from the major manufacturers (Zeiss, Leitz, Olympus, and Nikon) is suitable for gene-specific FISH analysis; the 60X Plan Apos or other objectives in which chromatic aberrations are carefully corrected are preferred. The number of test and reference probe signals is evaluated from a minimum of 100 randomly chosen nuclei throughout the slide. Only morphologically intact and nonoverlapping nuclei are counted. Because the nuclei are three-dimensional, it is necessary to move the focus throughout the depth of the nuclei to obtain the correct signal count.

Several formats are typically used for reporting the results of gene-specific FISH, for example: (1) the number of test probe signals per cell; (2) the number of signals per cell from the test probe divided by those from the reference probe; or (3) the percentage of cells where the test probe signal number is present at a higher or lower copy number than the reference probe.

Please refer to FIG. 9A-9E. FIG. 9A-9E illustrates the principle by which two-color FISH analysis with a CD16a receptor gene-specific test probe labeled in one color and a reference probe labeled in another color can be applied to detect transgenic, synthetic, genetically modified, or deliberately delivered DNA sequence encoding the CD 16a receptor in human natural killer cells.

Based on applicant's experience, the number of FCGR3A FISH Probe (a test probe which could detect all of the human DNA sequence encoding CD16a receptor) signals per oNK cell would be 2 (the actual gene copy number per cell), and two-color FISH pattern of oNK would look like FIG. 9A (normal pattern indicating the result of a human cell with no transgenic, synthetic, genetically modified, or deliberately delivered DNA sequence encoding the CD 16a receptor). The number of Chromosome 1 Control Probe (a reference probe; Purchased from Empire Genomics) signals per yNK cell may be larger than 2, and two-color FISH pattern of yNK would look like FIG. 9B-9E (CD16-transgenic pattern indicating the result of a human cell with transgenic, synthetic, genetically modified, or deliberately delivered DNA sequence encoding the CD 16a receptor).

### Embodiment 9: Effect of Freezing and Thawing on the Survival Rate of Non-Transgenic Human CD16⁺ Natural Killer Cell Line

The purified CD16⁺ cell population (the proportion of cells expressing CD16 receptor was as high as 99%) was sorted by the method of Embodiment 1.1, and then the purified CD16⁺ cell population was cultured for 21 days by the culture method of Embodiment 2.1 (purified CD CD16⁺ cell population was subcultured for 8 times). The sample of the cell solution was mixed with an equal volume of Trypan blue, then subjected to cell count and learned that the cell survival rate is 95%. Take a sufficient amount of the cell solution that contained 2×10⁷ viable cells, then perform the following freezing and thawing procedures.

Freezing procedure: centrifuged the cell solution containing 2×10⁷ viable cells, and removed the supernatant then resuspend the cell using 1 mL of frozen medium (CryoStor® CS10 Freeze Media, containing 10 vol % DMSO, BioLife Solutions, USA). The cell suspension was placed in a cryotube, and the cryotube was placed in the CoolCell Cell freezing container (Corning, USA), then stored the CoolCell Cell freezing container in a −80° C. refrigerator overnight (which decreased 1° C. per minute). The cryotube was transferred and stored in liquid nitrogen for 17 days.

Thawing procedure: place the cryotube in a 37° C. water bath to quickly thaw the cell suspension, and mix 1 mL of cell suspension with 9 mL of cell culture medium in Embodiment 2.1. After mixing a sample of the cell mixture with an equal volume of Trypan blue, the cell number and cell viability were observed.

The experimental results showed that 1.95×10⁷ cell survived after thawing, and the Recovery rate was as high as 97.5%[(1.95×10⁷)÷(2×10⁷)×100%=97.5%], and the cell survival rate was 96% that had no significant difference from viability (95%) before freezing.

### Embodiment 10: Cytotoxic Activity of Non-Transgenic Human CD16⁺ Natural Killer Cell Line

The experimental method of this embodiment is almost the same as that of Embodiment 3.5, except that (1) the effector cell used in this embodiment is Ctrl oNK cells, Ctrl yNK cells, ACE-oNK cells, or ACE-yNK cells; and (2) the ratio of the number of effector cells to the number of SKOV-3 cells (target cells) is 2:1 (ET2) or 5:1 (ET5).

Ctrl oNK cell: Ctrl oNK cells are the cultured cell population after the purified CD16⁺ cell populations (wherein the proportion of non-transgenic human CD16⁺ natural killer cell line is as high as 99%) were cultured for 26 days by using the method of Embodiment 2.1.

Ctrl yNK cell: Ctrl yNK cells are CD16-transgenic NK-92 cell line (Purchased from ATCC; The deposit number is ATCC PTA-6967);

ACE-oNK cell: ACE-oNK cells are cells obtained by binding Trastuzumab to Ctrl oNK cells using a cell linker and a Trastuzumab linker that are complementary.

ACE-yNK cell: ACE-yNK cells are cells obtained by binding Trastuzumab (an antibody against HER2 protein, product name as Herceptin, purchased from Roche Swiss) to Ctrl yNK cells using a cell linker and a Trastuzumab linker that are complementary.

The procedure of binding Trastuzumab to natural killer cells (e.g., Ctrl oNK cells or Ctrl yNK cells) are as follows: (A) The step of preparing cell linker and binding the cell linker to the natural killer cell in order to prepare an NK-ssDNA conjugate; (B) The step of preparing Trastuzumab linker and binding the Trastuzumab linker to Trastuzumab in order to prepare the Trastuzumab-ssDNA conjugate; (C) Mixing NK-ssDNA conjugate and Trastuzumab-ssDNA conjugate to combine NK-ssDNA conjugate and Trastuzumab-ssDNA conjugate through the cell linker and its complementary sequence on the Trastuzumab linker in order to prepare Trastuzumab-conjugated natural killer cells (e.g., ACE-oNK cells or ACE-yNK cells).

Wherein the step (A) of preparing cell linker and binding the cell linker to the natural killer cell comprises the following steps (a1)˜(a4):

Step (a1) A first single strand DNA was obtained, wherein the sequence of the first single strand DNA was SEQ ID NO:5, SEQ ID NO:6, or SEQ ID NO:7.

Step (a2) The 5′ end of the first single strand DNA was modified as 5′ end thiol-modified first single strand DNA to obtain the cell linker stock. The cell linker stock is also commercially available from Integrated DNA Technologies. Actual methods of modification are known, or will be apparent, to those skilled in the art (Zimmermann, J, 2010).

Step (a3) 10-500 μL cell linker stock and 0.1-10 μL NHS-Maleimide (commercially available from Fisher Scientific) were mixed and incubated for 1-60 minute(s).

Step (a4) The mixture obtained from Step (a3) were mixed with 1×10⁶-1×10⁸ natural killer cells and incubated for 1-60 minutes to obtain NK-ssDNA conjugate.

the step (B) of preparing Trastuzumab linker and binding the Trastuzumab linker to Trastuzumab comprises the following steps (b1)˜(b4):

Step (b1) A second single strand DNA was obtained, wherein the sequence of the second single strand DNA was SEQ ID NO:8, SEQ ID NO:9, or SEQ ID NO:10, and the sequence of the second single strand DNA is the complementary strand to the first single strand DNA.

Step (b2) The 5′ end of the second single strand DNA was modified as 5′ end thiol-modified second single strand DNA to obtain the Trastuzumab linker stock. The Trastuzumab linker stock is also commercially available from Integrated DNA Technologies. Actual methods of modification are known, or will be apparent, to those skilled in the art (Zimmermann, J, 2010).

Step (b3) 10-500 μL Trastuzumab linker stock and 0.1-10 μL NHS-Maleimide (commercially available from Fisher Scientific) were mixed and incubated for 1-60 minute(s).

Step (b4) The mixture obtained from Step (b3) were mixed with 10-100 μL Trastuzumab stock (commercially available from Roche) and incubated for 10 minutes to 3 hours to obtain Trastuzumab-ssDNA conjugate.

Please refer to FIG. 10. FIG. 10 is the bar chart presenting the cytotoxic function of non-transgenic human CD16⁺ natural killer cell line to kill cancer cells through ADCC process. FIG. 10 shows that regardless of the ratio of the number of effector cells to the number of SKOV-3 cells (target cells) is 2:1 (ET2) or 5:1 (ET5), non-transgenic human CD16⁺ natural killer cell lines (Ctrl oNK cells) that were not activated by Trastuzumab killed 60%˜65% of cancer cells, whereas Trastuzumab-activated non-transgenic human CD16⁺ natural killer cell line (ACE-oNK cells) killed 95%˜100% of cancer cells. Thus, the non-transgenic human CD16⁺ natural killer cell line obtained by the culture of the present invention indeed have the cytotoxic function to kill cancer cells, and when the non-transgenic human CD16⁺ natural killer cell line obtained by the culture of the present invention was activated to induce ADCC reaction, the cytotoxic effect was significantly increased by at least 30% (95%-65%=30%; p<0.05).

Please refer to FIGS. 11A and 11B. FIG. 11A is the bar chart presenting the comparison of the cytotoxic function between the non-transgenic human CD16⁺ natural killer cell line and CD16-transgenic NK-92 cell line to kill cancer cells at different effector (E) to target (T) ratio; and FIG. 11B is the bar chart presenting the comparison of the cytotoxic function between the non-transgenic human CD16⁺ natural killer cell line and CD16-transgenic NK-92 cell line to kill cancer cells through ADCC process at different effector (E) to target (T) ratio.

The results of FIG. 11A show that when the ratio of the number of effector cells to the number of SK-OV-3 cells (target cells) is 5:1 (ET5) and not activated by Trastuzumab, non-transgenic human CD16⁺ natural killer cell lines (Ctrl oNK cell) kill 70% of cancer cells, while CD16-transgenic NK-92 cell line (Ctrl yNK) kill 72% of cancer cells, there was no significant difference between the two groups (p>0.05). Thus, when the ratio of the number of effector cells to the number of SK-OV-3 cells (target cells) is 5:1 (ET5), the cytotoxic function of the non-transgenic human CD16⁺ natural killer cell line obtained by the culture of the present invention was not inferior to the CD16-transgenic NK-92 cell line. In other words, compared with the CD16-transgenic NK-92 cell line, the non-transgenic human CD16⁺ natural killer cell line obtained by the method of the present invention is not only safe but also has the same cytotoxic effect.

The results in FIG. 11B show that regardless of the ratio of the number of effector cells to the number of SK-OV-3 cells (target cells) is 2:1 (ET2) or 5:1 (ET5), Trastuzumab-activated non-transgenic human CD16⁺ natural killer cell line (ACE-oNK cells) killed 95% of cancer cells, whereas Trastuzumab-activated CD16-transgenic NK-92 cell line (ACE-yNK) also killed 95% of cancer cells, and there was no significant difference between the two groups (p>0.05). Thus, the cytotoxic function through ADCC process of the non-transgenic human CD16⁺ natural killer cell line obtained by the culture method of the present invention was not inferior to the CD16-transgenic NK-92 cell line. In other words, compared with the CD16-transgenic NK-92 cell line, the non-transgenic human CD16⁺ natural killer cell line obtained by the method of the present invention is not only safe, but also had the same cytotoxic effect in killing cancer cells through ADCC process.

### Embodiment 11: Culturing Non-Transgenic Human CD16⁺ Natural Killer Cell Line with Different Concentration of Human Platelet Lysate

The experimental method of this embodiment is almost the same as that of Embodiment 2.1, except that (1) in Step S22′, all of the cells in the cell suspensions obtained by culturing for 9 days with the culture method disclosed in the embodiments 2.1 (refer to as 9-day cultured oNK suspension) were cultured in this embodiment and the number of cells in the first container in Step S22′ was 5×10⁶; and (2) the cell culture medium comprises 500 IU/mL IL-2 and \textcircled{1} 2.5% human platelet lysate, \textcircled{2} 5.0% human platelet lysate, \textcircled{3} 10.0% human platelet lysate, or \textcircled{4} 5.0% human serum (comprising no human platelet lysate).

The experimental method of detecting cell number, cell viability, and CD16 surface marker of the cultured cells in this embodiment is the same as that of Embodiment 2.2 and 3.4.

Please refer to FIG. 12A-12C. FIG. 12A-12C are the line graph presenting the effect of human platelet lysate on total cell number, cell viability, or maintaining the expression of CD16 respectively after different days of culturing human CD16⁺ natural killer cell line.

FIG. 12A showed that after culturing for 14 days, the number of the non-transgenic human CD16⁺ natural killer cells cultured in cell culture medium comprising no human platelet lysate (but comprising 5.0% human serum), 2.5% human platelet lysate, 5.0% human platelet lysate, and 10.0% human platelet lysate were 4.7×10⁸, 6.49×10⁸, 1.01×10⁹, and 1.74×10⁹ respectively. Thus, the result shows that: as compare with cell culture medium comprising no human platelet lysate (but comprising 5.0% human serum), human platelet lysate could cause 3.7 fold increase (17.4÷4.7=3.7). That is, human platelet lysate makes an unexpected result, and human platelet lysate makes non-transgenic human CD16⁺ natural killer cells expand greatly. Moreover, these results suggested that Formula 3 (comprising 10.0% human platelet lysate) was better than the rest of formulas for human CD16⁺ natural killer cells expansion.

FIG. 12B showed that after culturing for 7 days, the cell viability of the non-transgenic human CD16⁺ natural killer cells cultured in cell culture medium comprising no human platelet lysate (but comprising 5.0% human serum), 2.5% human platelet lysate, 5.0% human platelet lysate, and 10.0% human platelet lysate were maintained at 92%, 88%, 92%, and 92% respectively. After culturing for 14 days, the cell viability of the non-transgenic human CD16⁺ natural killer cells cultured in cell culture medium comprising no human platelet lysate (but comprising 5.0% human serum), 2.5% human platelet lysate, 5.0% human platelet lysate, and 10.0% human platelet lysate were maintained at 94%, 90%, 92%, and 93% respectively. Thus, the result shows that: human CD16⁺ natural killer cells did not be treated with human platelet lysate have similar viability as human CD16⁺ natural killer cells treated with 2.5%-10.0% human platelet lysate.

FIG. 12C showed that after culturing in cell culture medium comprising no human platelet lysate (but comprising 5.0% human serum), 2.5% human platelet lysate, 5.0% human platelet lysate, or 10.0% human platelet lysate for 7 days, the percentage of CD16⁺ cells were maintained at 83.55%, 84.15%, 82.81%, and 83.95% respectively. After culturing in cell culture medium comprising no human platelet lysate (but comprising 5.0% human serum), 2.5% human platelet lysate, 5.0% human platelet lysate, or 10.0% human platelet lysate for 14 days, the percentage of CD16⁺ cells were maintained at 80.72%, 80.74%, 78.07%, and 80.76% respectively. Thus, the result shows that: 2.5%-10% human platelet lysate maintains similar CD16⁺ population as no human platelet lysate (comprising 5.0% human serum).

Moreover, based on this result, applicant believes that after isolating human CD16⁺ natural killer cell line from the 9-day cultured oNK suspension (cultured oNK), similar result could be observed.

### Embodiment 12: Culturing Non-Transgenic Human CD16⁺ Natural Killer Cell Line with Different Concentration of IL-2

The experimental method of this embodiment is almost the same as that of Embodiment 2.1, except that (1) in Step S22′, all of the cells in the cell suspensions obtained by culturing for 9 days with the culture method disclosed in the embodiments 2.1 (refer to as 9-day cultured oNK suspension) were cultured in this embodiment and the number of cells in the first container in Step S22′ was 5×10⁶; and (2) the cell culture medium comprises 5.0% human platelet lysate and \textcircled{1} 100 IU/mL IL-2, \textcircled{2} 200 IU/mL IL-2, 500 IU/mL IL-2, 750 IU/mL IL-2, or 1000 IU/mL IL-2.

Please note that both of IL-2 and human platelet lysate were required for expansion human CD16⁺ natural killer cells. In this embodiment, 1.8×10⁷ IU/mL IL-2 was equal to 1.1 mg/mL IL-2. Therefore, 100 IU/mL IL-2 was equal to 0.0612 μg/mL IL-2; 200 IU/mL IL-2 was equal to 0.1224 μg/mL IL-2; 500 IU/mL IL-2 was equal to 0.306 μg/mL IL-2; 750 IU/mL IL-2 was equal to 0.459 μg/mL IL-2; and 1000 IU/mL IL-2 was equal to 0.612 μg/mL IL-2.

The experimental method of detecting cell number, cell viability, and CD16 surface marker of the cultured cells in this embodiment is the same as that of Embodiment 2.2 and 3.4.

Please refer to FIG. 13A-13F. FIG. 13A-13F are the line graph presenting the effect of IL-2 on total cell number, cell viability, or maintaining the expression of CD16 respectively after different days of culturing human CD16⁺ natural killer cell line.

FIG. 13A-13B showed that IL-2 level did not influence on non-transgenic human CD16⁺ natural killer cell expansion. Please note that cells were reseeded on Day 7 and then continued to expand to Day 11; the expansion process was repeated every 11 days.

FIG. 13C-13D showed that IL-2 level did not influence on cell viability of the non-transgenic human CD16⁺ natural killer cells.

FIG. 13E-13F showed that after culturing in cell culture medium comprising 100-200 IU/mL IL-2 for 40 days, the percentage of CD16⁺ cells was dropped to less than 20%. On the other hand, after culturing in cell culture medium comprising 500-1000 IU/mL IL-2 for 40 days, the percentage of CD16⁺ cells was increased to 80%. That is, 500-1000 IU/mL IL-2 makes an unexpected result, and 500-1000 IU/mL IL-2 makes CD16⁺ population maintain greatly.

Moreover, based on this result, applicant believes that after isolating human CD16⁺ natural killer cell line from the 9-day cultured oNK suspension (cultured oNK), similar results could be observed.

### Embodiment 13: Culturing Non-Transgenic Human CD16⁺ Natural Killer Cell Line in Different Container

The experimental method of this embodiment is almost the same as that of Embodiment 2.1, except that (1) in Step S22′, all of the cells in the cell suspensions obtained by culturing for 9 days with the culture method disclosed in the embodiments 2.1 (refer to as 9-day cultured oNK suspension) were cultured in this embodiment and the number of cells in the first container in Step S22′ was 5×10⁶; (2) the cell culture medium comprises 500 IU/mL IL-2 and 5.0% human platelet lysate; and (3) the containers used in this embodiment are \textcircled{1} air-permeable container such as G-Rex 6-well culture plate or \textcircled{2} non air-permeable container such as T25 cell culture flask.

The experimental method of detecting cell number, cell viability, and CD16 surface marker of the cultured cells in this embodiment is the same as that of Embodiment 2.2 and 3.4.

Please refer to FIG. 14A-14C. FIG. 14A-14C are the line graph presenting the effect of air-permeable container on total cell number, cell viability, or maintaining the expression of CD16 respectively after different days of culturing human CD16⁺ natural killer cell line.

FIG. 14A showed that after culturing for 14 days, the number of the non-transgenic human CD16⁺ natural killer cells cultured in non air-permeable container and air-permeable container were 3.1×10⁸ and 1.01×10⁹ respectively. Thus, the result shows that: as compare with cells cultured in non air-permeable container, air-permeable container could cause 3.26 fold increase (10.1÷3.1=3.26). That is, air-permeable container makes an unexpected result, and air-permeable container makes non-transgenic human CD16⁺ natural killer cells expand greatly.

FIG. 14B showed that after culturing for 7 days, the cell viability of the non-transgenic human CD16⁺ natural killer cells cultured in non air-permeable container and air-permeable container were maintained at 87% and 92% respectively. After culturing for 14 days, the cell viability of the non-transgenic human CD16⁺ natural killer cells cultured in non air-permeable container and air-permeable container were maintained at 88% and 92% respectively. Thus, the result shows that: human CD16⁺ natural killer cells cultured in air-permeable container better viability than human CD16⁺ natural killer cells cultured in non air-permeable container.

FIG. 14C showed that after culturing in non air-permeable container and air-permeable container for 7 days, the percentage of CD16⁺ cells were maintained at 82.63% and 82.81% respectively. After culturing in non air-permeable container and air-permeable container for 14 days, the percentage of CD16⁺ cells were maintained at 83.79% and 88.07% respectively. Thus, the result shows that: air-permeable container maintains similar CD16⁺ population as non air-permeable container does.

Moreover, based on this result, applicant believes that after isolating human CD16⁺ natural killer cell line from the 9-day cultured oNK suspension (cultured oNK), similar result could be observed.

### Embodiment 14: Prepare Exogenous Targeting Unit Complexed-oNK Cells

In this embodiment, applicant prepares an exogenous targeting unit complexed-oNK cell to which at least an exogenous targeting unit complexed. The exogenous targeting unit comprises an targeting moiety which exhibits specific binding to a biological marker on a target cell, and the targeting moiety could bind to a biological marker selected form cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, or MAGE. The targeting moiety is not a nucleic acid and is not produced by the exogenous targeting unit complexed-oNK cell.

The procedure of binding a targeting moiety (such as Trastuzumab which is against HER2 protein) to oNK cells are as follows: (A) The step of preparing cell linker and binding the cell linker to the natural killer cell in order to prepare an NK-ssDNA conjugate; (B) The step of preparing targeting moiety linker (such as Trastuzumab linker) and binding the targeting moiety linker to the targeting moiety in order to prepare the targeting moiety-ssDNA conjugate; (C) Mixing NK-ssDNA conjugate and targeting moiety-ssDNA conjugate to combine NK-ssDNA conjugate and targeting moiety-ssDNA conjugate through the cell linker and its complementary sequence on the targeting moiety linker in order to prepare exogenous targeting unit complexed-conjugated natural killer cells (e.g., ACE-oNK cells or ACE-yNK cells).

Wherein the step (A) of preparing cell linker and binding the cell linker to the natural killer cell comprises the following steps (a1)˜(a4):

Step (a1) A first single strand DNA was obtained, wherein the sequence of the first single strand DNA was SEQ ID NO:5, SEQ ID NO:6, or SEQ ID NO:7.

Step (a2) The 5′ end of the first single strand DNA was modified as 5′ end thiol-modified first single strand DNA to obtain the cell linker stock. The cell linker stock is also commercially available from Integrated DNA Technologies. Actual methods of modification are known, or will be apparent, to those skilled in the art (Zimmermann, J, 2010).

Step (a3) 10-500 μL cell linker stock and 0.1-10 μL NHS-Maleimide (commercially available from Fisher Scientific) were mixed and incubated for 1-60 minute(s).

Step (a4) The mixture obtained from Step (a3) were mixed with 1×10⁶-1×10⁸ natural killer cells and incubated for 1-60 minutes to obtain NK-ssDNA conjugate.

The step (B) of preparing targeting moiety linker and binding the targeting moiety linker to targeting moiety comprises the following steps (b1)˜(b4):

Step (b1) A second single strand DNA was obtained, wherein the sequence of the second single strand DNA was SEQ ID NO:8, SEQ ID NO:9, or SEQ ID NO:10, and the sequence of the second single strand DNA is the complementary strand to the first single strand DNA.

Step (b2) The 5′ end of the second single strand DNA was modified as 5′ end thiol-modified second single strand DNA to obtain the targeting moiety linker stock. The targeting moiety linker stock is also commercially available from Integrated DNA Technologies. Actual methods of modification are known, or will be apparent, to those skilled in the art (Zimmermann, J, 2010).

Step (b3) 10-500 μL targeting moiety linker stock and 0.1-10 μL NHS-Maleimide (commercially available from Fisher Scientific) were mixed and incubated for 1-60 minute(s). Step (b4) The mixture obtained from Step (b3) were mixed with 10-100 μL targeting moiety stock (commercially available from Roche) and incubated for 10 minutes to 3 hours to obtain targeting moiety-ssDNA conjugate.

The targeting moiety could be a peptide, protein, or aptamer, wherein the protein could be an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, activating NK cell receptors, and combinations thereof.

### Embodiment 15: Prepare Chimeric Antigen Receptor (CAR)-Expressed oNK Cells

The method for preparing oNK cells comprising a synthetic, genetically modified and/or deliberately delivered polynucleotide encoding a chimeric antigen receptor (CAR) comprising a target-binding single-chain variable fragment (scFv) against target antigen is disclosed in this embodiment, wherein the chimeric antigen receptor is selected from CD2, CD3 delta, CD3 epsilon, CD3 gamma, CD4, CD7, CD8a, CD8, CD1 1a (ITGAL), CD1 1b (ITGAM), CD1 1c (ITGAX), CD1 1d (ITGAD), CD 18 (ITGB2), CD 19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD1 1a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2, BCMA, PD-L1, and combinations thereof.

Take CD19 as an example to explain the method of preparing oNK comprising a synthetic, genetically modified and/or deliberately delivered polynucleotide encoding a chimeric antigen receptor (CAR) comprising a target-binding single-chain variable fragment (scFv) against CD19 as below. oNK cells were harvested and treated with 6 μM of 5Z-7-Oxozeaenol (TAK1 inhibitor) for 30 min. The 5Z-7-Oxozeaenol-treated cells were treated with Trasdux (transduction enhancer) and infected with lentivirus particles produced by anti-CD19 scFv CAR-expressing construct in CD810A-1 vector at m.o.i. of 9. The cells were centrifuged at 1000×g for 70 min, and then incubated at 37° C. for 4 hr. The cells were further centrifuged at 200×g for 5 min and the supernatant was removed. The infected cells were resuspended with fresh growth media and cultured at 37° C. in G-Rex plate.

To enrich CAR-expressing populations, the cells were centrifuged at 400×g for 5 min and washed by MACS buffer. The cells were further stained with anti-Myc Ab-APC and DAPI. After washed by DPBS, the APC⁺ DAPI⁻ cells were sorted out by cell sorter and then expanded in fresh growth media in G-Rex.

From the embodiments of the present invention, it demonstrated that all of the non-transgenic human CD16⁺ natural killer cell line obtained by the method of the present invention, the exogenous targeting unit complexed-natural killer cell of the present invention, and the chimeric antigen receptor (CAR)-expressed oNK cells of the invention can indeed kill the target cell (e.g., cancer cells) though ADCC-like process. Therefore, the applicable fields of the non-transgenic human CD16⁺ natural killer cell line, obtained by the culture method of the present invention, include but not limited to cancer treatment, autoimmune disease treatment, neuronal disease treatment, human immunodeficiency virus (HIV) eradication, hematopoietic cell-related diseases, metabolic syndrome treatment, pathogenic disease treatment, treatment of viral infection, and treatment of bacterial infection.
Reference 1—Eileen Scully and Galit Alter, 2016. NK cells in HIV disease. Curr HIV/AIDS Rep. 13(2):85-94.
Reference 2—Jordan S. Orange, 2013. Natural killer cell deficiency. J Allergy Clin Immunol. 132(3):515-525.
Reference 3—Kallioniemi A, Visakorpi T, Karlui R, Pinkel D, and Kallioniemi OP, 1996. Gene Copy number analysis by fluorescence in situ hybridization and comparative genomic hybridization. Methods. 9(1):113-121.
Reference 4—Littwitz-Salomon E, Dittmer U, Sutter K, 2016. Insufficient natural killer cell responses against retroviuses: how to improve NK cell killing of retrovirus-infected cells. Retrovirology. 13(1):77.
Reference 5—Pernick, N, 2018.

http://www.pathologyoutlines.com/topic/cdmarkerscd56.html

http://www.pathologyoutlines.com/topic/cdmarkerscd3.html

http://www.pathologyoutlines.com/topic/cdmarkerscd2.html

http://www.pathologyoutlines.com/topic/cdmarkerscd16.html
Reference 6—Rezvani K and Rouce RH, 2015. The application of natural killer cell immunotherapy for the treatment of cancer. Front Immunol. 6:578.
Reference 7—Vindelov, L. L., Christensen, I. J., and Nissen, N. I., 1983. A Detergent-trypsin method for the preparation of nuclei for flow cytometric DNA analysis. Cytometry. 3(5), 323-327.
Reference 8—Zimmermann, J, Nicolaus, T, Neuert, G. and Blank, K. 2010. Thiol-based, site-specific and covalent immobilization of biomolecules for single-molecule experiments. Nat. Protoc. 5(6):975-985.

The foregoing descriptions are merely the preferred embodiments of the present invention and are not intended to limit the scope of the patent application of the present invention. Therefore, any alteration or modification that does not depart from the spirits disclosed herein should be included within the scope of the patent application of the present invention.

1-82. (canceled)

83. A human natural killer cell which is derived from a subject with a cancer and has the following characteristics:
i) expressing a CD16 receptor;
ii) retaining its capability to proliferate after subculture for at least 3 months; and
iii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

84. The cell according to claim 83, wherein the CD16 receptor is a CD16a receptor or a CD16b receptor.

85. The cell according to claim 83, wherein an expressed polynucleotide encoding the CD16 receptor is located on q arm of chromosome 1 at position 1q23.3.

86. The cell according to claim 83, the cell is non-tumorigenic in an immune compromised mouse.

87. The cell according to claim 83, wherein, after being irradiated with γ-ray, the cell is non-tumorigenic in an allogeneic subject.

88. The cell according to claim 83, wherein a polynucleotide encoding the CD16 receptor comprising a nucleotide sequence of SEQ NO:1, SEQ ID NO:2, or SEQ ID NO:19.

89. The cell according to claim 83, wherein the CD16 receptor comprising an amino acid sequence of SEQ ID NO:3, SEQ ID NO:4, or SEQ ID NO:20.

90. The cell according to claim 83, the cell further comprises an inactive tumor suppressor gene or a mutated and highly expressed oncogene.

91. The cell according to claim 83, wherein the cell is capable of mediating an antibody-dependent cell cytotoxicity (ADCC) response, and the cell is a male cell.

92. The cell according to claim 83, wherein the cell further comprises at least an exogenous targeting unit complexed to the cell, wherein the exogenous targeting unit comprises a targeting moiety that is characterized in that:
(a) it exhibits specific binding to a biological marker on a target cell;
(b) it is not a nucleic acid; and
(c) it is not produced by the cell.

93. The cell according to claim 92, wherein the exogenous targeting unit is complexed to the cell via an interaction between a first linker conjugated to the targeting moiety and a second linker conjugated to the cell.

94. The cell of claim 93, wherein the first linker is a first polynucleotide, or the second linker is a second polynucleotide.

95. The cell of claim 92, wherein the targeting moiety comprises an antigen-binding unit.

96. The cell of claim 94, wherein the first polynucleotide comprises a single-stranded region.

97. The cell of claim 93, wherein the first linker is a first polynucleotide, and the second linker is a second polynucleotide.

98. The cell of claim 93, wherein the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

99. The cell of claim 93, wherein the first linker comprises a first reactive group, and the second linker comprises a second reactive group, and wherein the cell is complexed to the targeting moiety via a covalent bond formed by a reaction between the second reactive group and the first reactive group.

100. The cell of claim 97, wherein the targeting moiety comprises an antigen-binding unit.

101. The cell of claim 93, wherein the second linker comprises a PEG region.

102. The cell of claim 92, wherein the targeting moiety and the cell are separated by a length of 1 nm to 400 nm.

103. The cell of claim 92, wherein the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multi drug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, or MAGE.

104. The cell of claim 103, wherein the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), CD105 (Endoglin), SIR, CD47, CEA, IL-17A, DLL4, CD51, angiopoietin 2, neuropilin-1, CD37, CD223 (LAG-3), CD40, (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNT receptor protein, an immunoglobulin protein, a cytokine receptor, an integrin, activating NK cell receptors, and combinations thereof.

105. The cell of claim 94, wherein the targeting moiety is conjugated to the first polynucleotide using a coupling group, wherein the coupling group is an NHS ester, other activated ester, an alkyl or acyl halide, a bifunctional crosslinker, or maleimide group.

106. The cell of claim 94, wherein the firm polynucleotide or second polynucleotide comprise a sequence selected from 20-mer poly-CA, 20-mer poly-GGTT, SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ II) NO: 25, SEQ II) NO: 26, SEQ ID NO: 27, SEQ ID NO: 28, SEQ NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7. and SEQ 1D NO:10.

107. The cell of claim 92, the binding affinity of the targeting moiety for the biological marker is less than 250 nM.

108. The cell of claim 94, the length of the first polynucleotide or the length of the second polynucleotide are 4 nt to 500 nt.

109. The cell of claim 93, the binding affinity between the first linker and the second linker is less than 250 nM.

110. The cell of claim 93, the first linker or the second linker is conjugated to a native functional group of the targeting unit or a surface of the cell, wherein the native functional group is an amino acid, a sugar, or an amine.

111. The cell of the claim 92, the targeting moiety is a peptide, protein, or aptamer.

112. A composition substantially enriched in human CD16⁺ natural killer cells, wherein the number of the human CD16⁺ natural killer cells in the composition is at least 5×10⁵ and the human CD16⁺ natural killer cells are in an amount equal to or more than 5% by number, based on the total number of the cells in the composition as 100%; the human CD16⁺ natural killer cell is derived from a subject with a cancer and has the following characteristics:
i) expressing a CD16 receptor,
ii) retaining its capability to proliferate after subculture for at least 3 months, and
iii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

113. The composition according to claim 112, wherein the CD16 receptor is a CD16a receptor or a CD16b receptor.

114. The composition according to claim 112, wherein a polynucleotide encoding the CD16 receptor is located on q arm of chromosome 1 at position 1q23.3.

115. The composition according to claim 112, wherein the human CD16⁺ natural killer cells are non-tumorigenic in an immune compromised mouse.

116. The composition according to claim 112, wherein, after being irradiated with γ-ray, the human CD16⁺ natural killer cells are non-tumorigenic in an allogeneic subject.

117. The composition according to claim 112, wherein a polynucleotide encoding the CD16 receptor comprises a nucleotide sequence of SEQ ID NO:1, SEQ ID NO:2, or SEQ ID NO:19.

118. The composition according to claim 112, wherein the CD16 receptor comprising an amino acid sequence of SEQ ID NO:3, SEQ ID NO:4, or SEQ ID NO:20.

119. The composition according to claim 112, wherein the human CD16⁺ natural killer cell further comprises an inactive tumor suppressor gene or a mutated and highly expressed oncogene.

120. The composition according to claim 112, wherein the human CD16⁺ natural killer cell is capable of mediating an antibody-dependent cell cytotoxicity (ADCC) response, and the cell is a male cell.

121. The composition according to claim 112, wherein the human CD16⁺ natural killer cell further comprises at least an exogenous targeting unit complexed to the human CD16⁺ natural killer cell, wherein the exogenous targeting unit comprises a targeting moiety that is characterized in that:
(a) it exhibits specific binding to a biological marker on a target cell;
(b) it is not a nucleic acid; and
(c) it is not produced by the human CD16⁺ natural killer cell.

122. The composition according to claim 121, wherein the exogenous targeting unit is complexed to the human CD16⁺ natural killer via an interaction between a first linker conjugated to the targeting moiety and a second linker conjugated to the human CD16⁺ natural killer cell.

123. The composition of claim 122, wherein the first linker is a first polynucleotide, or the second linker is a second polynucleotide.

124. The composition of claim 121, wherein the targeting moiety comprises an antigen-binding unit.

125. The composition of claim 123, wherein the first polynucleotide comprises a single-stranded region.

126. The composition of claim 122, wherein the first linker is a first polynucleotide, and the second linker is a second polynucleotide.

127. The composition of claim 122, wherein the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

128. The composition of claim 122, wherein the first linker comprises a first reactive group, and the second linker comprises a second reactive group, and wherein the human CD16⁺ natural killer cell is complexed to the targeting moiety via a covalent bond formed by a reaction between the second reactive group and the first reactive group.

129. The composition of claim 126, wherein the targeting moiety comprises an antigen-binding unit.

130. The composition of claim 122, wherein the second linker comprises a PEG region.

131. The composition of claim 121, wherein the targeting moiety and the human CD16⁺ natural killer cell is separated by a length of 1 nm to 400 nm.

132. The composition of claim 121, wherein the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, or MAGE.

133. The composition of claim 132, wherein the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, activating NK cell receptors, and combinations thereof.

134. The composition of claim 123, wherein the targeting moiety is conjugated to the first polynucleotide using a coupling group, wherein the coupling group is an NHS ester, other activated ester, an alkyl or acyl halide, a bifunctional crosslinker, or maleimide group.

135. The composition of claim 123, wherein the first polynucleotide or second polynucleotide comprise a sequence selected from 20-mer poly-CA, 20-mer SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7, and SEQ ID NO:10.

136. The composition of claim 121, the binding affinity of the targeting moiety for the biological marker is less than 250 nM.

137. The composition of claim 123, the length of the first polynucleotide or the length of the second polynucleotide are 4 nt to 500 nt.

138. The composition of claim 122, the binding affinity between the first linker and the second linker is less than 250 nM.

139. The composition of claim 122, the first linker or the second linker is conjugated to a native functional group of the targeting unit or a surface of the human CD16⁺ natural killer cell, wherein the native functional group is an amino acid, a sugar, or an amine.

140. The composition of the claim 121, the targeting moiety is a peptide, protein, or aptamer.

141. A method of obtaining a composition substantially enriched in human CD16⁺ natural killer cells; the method comprising:
(a) obtaining a population of human peripheral blood natural killer cells having the deposit number ATCC CRL-2407;
(b) contacting the population of human peripheral blood natural killer cells with an antibody specific for a CD16 receptor; and
(c) separating cells that are specifically bound by the antibody thereby obtaining the composition substantially enriched in human CD16⁺ natural killer cells;
wherein the human CD16⁺ natural killer cell is:
(A) deposited at NPMD having the deposit number NIT EE BP-03017; or
(B) having the following characteristics:
i) expressing a CD16 receptor, and
ii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

142. The method according to claim 141, wherein the step (c) comprises substeps:
(c1) separating cells that are specifically bound by the antibody;
(c2) in a container, contacting the cells that are specifically bound by the antibody with a culture medium comprising human platelet lysate and IL-2; and
(c3) culturing the cells for multiple days thereby obtaining the composition substantially enriched in human CD16⁺ natural killer cells.

143. The method according to claim 142, wherein the container comprises a bottom for seeding cells, and the bottom is air-permeable and water-impermeable.

144. The method according to claim 142, wherein the concentration of the dissolved glucose in the culture medium is 1500-5000 mg/L.

145. The method according to claim 142, the number of the human CD16⁺ natural killer cells in the composition is at least 5×10⁵, and the human CD16⁺ natural killer cells are in an amount equal to or more than 5% by number, based on the total number of the cells in the composition as 100%.

146. A method of culturing and expanding human CD16⁺ natural killer cells; the method comprising
(x) in a container, contacting the human CD16⁺ natural killer cells with a culture medium comprising 0.5-10 vol % human platelet lysate and 100-3000 IU/mLIL-2; and
(y) culturing the cells for multiple days.

147. The method according to claim 146, wherein the container comprises a bottom for seeding cells, and the bottom is air-permeable and water-impermeable.

148. The method according to claim 146, wherein the step (y) comprises substeps:
(y1) culturing the cells for at least one day; and
(y2) sub-culturing the cells for at least 1 months.

149. The method according to claim 146, wherein the human CD16⁺ natural killer cells are capable of retaining their capability to proliferate after subculture for at least 3 months.

150. The method according to claim 146, wherein the human CD16⁺ natural killer cell is:
(A) deposited at NPMD having the deposit number NITE BP-03017; or
(B) having the following characteristics:
i) expressing a CD16 receptor, and
ii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

151. A method of treating cancer, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of the human natural killer cell of claim 83 to a subject in need thereof.

152. The method according to claim 151, wherein the exogenous targeting unit is complexed to the human natural killer cell via an interaction between a first linker conjugated to the targeting moiety and a second linker conjugated to the human natural killer cell.

153. The method of claim 151, wherein the exogenous targeting unit comprises an antigen-binding unit.

154. The method of claim 152, wherein the antigen-binding unit is an antibody against a cancer antigen.

155. The method of the claim 151, the targeting moiety is a peptide, protein, or aptamer.

156. The method according to claim 151, wherein the human natural killer cell is capable of mediating an antibody-dependent cell cytotoxicity (ADCC) response, and the human natural killer cell is a male cell.

157. The method according to claim 151, wherein the method is for treating cancer.

158. The cell according to claim 83, wherein the cell further comprising a synthetic, genetically modified and/or deliberately delivered polynucleotide encoding a target-binding single-chain variable fragment (scFv) against an antigen.

159. The cell according to claim 157, wherein the cell is capable of mediating an antibody-dependent cell cytotoxicity (ADCC) response.

160. A method of treating cancer, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of the human natural killer cell of claim 157 to a subject in need thereof.

161. The method according to claim 159, wherein the number of the human natural killer cells in the composition is at least 5×10⁵ and the cells are in an amount equal to or more than 5% by number, based on the total number of the cells in the composition as 100%.

162. The method according to claim 159, wherein the method is for treating cancer.

163. A human natural killer cell which is deposited at NPMD having the deposit number NITE BP-03017.

164. A composition substantially enriched in human CD16⁺ natural killer cells, wherein the number of the human CD16⁺ natural killer cells in the composition is at least 5×10⁵ and the human CD16⁺ natural killer cells are in an amount equal to or more than 5% by number, based on the total number of the cells in the composition as 100%; the human CD16⁺ natural killer cell is deposited at NPMD having the deposit number NITE BP-03017.


SEQUENCE LISTING (41 sequences)

SEQ ID NO: 1 - DNA, 2137 bp, Homo sapiens
cttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 820 bp, Homo sapiens
cactccagtgtggcatcatgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcag

SEQ ID NO: 3 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 4 - protein, 233 aa, Homo sapiens
MWQLLLPTALLLLVSAGMRTEDLPKAVVFLEPQWYSVLEKDSVTLKCQGAYSPEDNSTQWFHSESLISSQASSYFIDAATVNESGEYRCQTNLSTLSDPVQLEVHIGWLLLRAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 5 - DNA, 20 bp, Artificial sequence
  synthetic
cacacacacacacacacaca

SEQ ID NO: 6 - DNA, 20 bp, Artificial sequence
  synthetic
tcatacgactcactctaggg

SEQ ID NO: 7 - DNA, 23 bp, Artificial sequence
  synthetic
agttaccatgacgtcaatttcag

SEQ ID NO: 8 - DNA, 20 bp, Artificial sequence
  synthetic
tgtgtgtgtgtgtgtgtgtg

SEQ ID NO: 9 - DNA, 20 bp, Artificial sequence
  synthetic
ccctagagtgagtcgtatga

SEQ ID NO: 10 - DNA, 23 bp, Artificial sequence
  synthetic
ctgaaattgacgtcatggtaact

SEQ ID NO: 11 - DNA, 51 bp, Artificial sequence
  synthetic
tctgaagacacatttttactcccaaaaagccccctgcagaagtaggagccg

SEQ ID NO: 12 - DNA, 51 bp, Artificial sequence
  synthetic
tctgaagacacatttttactcccaacaagccccctgcagaagtaggagccg

SEQ ID NO: 13 - DNA, 765 bp, Homo sapiens
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 14 - protein, 254 aa, Homo sapiens; identical to SEQ ID NO: 3

SEQ ID NO: 15 - DNA, 765 bp, Homo sapiens
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 16 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 17 - DNA, 51 bp, Artificial sequence
  synthetic
tccttcacaatttctgcagccactctgtggccaccgtcaccttattcctga

SEQ ID NO: 18 - DNA, 51 bp, Artificial sequence
  synthetic
tccttcacaatttctgcagccactccgtggccaccgtcaccttattcctga

SEQ ID NO: 19 - DNA, 765 bp, Homo sapiens; identical to SEQ ID NO: 13

SEQ ID NO: 20 - protein, 80 aa, Homo sapiens
HSWKNTALHKVTYLQNGKGRKYFHHNSDFYIPKATLKDSGSYFCRGLFGSKNVSSETVNITITQGLAVSTISSFFPPGYQ

SEQ ID NO: 21 - DNA, 20 bp, Artificial sequence
  synthetic
aaaaaaaaaaaaaaaaaaaa

SEQ ID NO: 22 - DNA, 20 bp, Artificial sequence
  synthetic
tttttttttttttttttttt

SEQ ID NO: 23 - DNA, 20 bp, Artificial sequence
  synthetic
actgactgactgactgactg

SEQ ID NO: 24 - DNA, 20 bp, Artificial sequence
  synthetic
cagtcagtcagtcagtcagt

SEQ ID NO: 25 - DNA, 20 bp, Artificial sequence
  synthetic
gtaacgatccagctgtcact

SEQ ID NO: 26 - DNA, 20 bp, Artificial sequence
  synthetic
agtgacagctggatcgttac

SEQ ID NO: 27 - DNA, 20 bp, Artificial sequence
  synthetic
actgatggtaatctgcacct

SEQ ID NO: 28 - DNA, 20 bp, Artificial sequence
  synthetic
aggtgcagattaccatcagt

SEQ ID NO: 29 - DNA, 40 bp, Artificial sequence
  synthetic
aaaaaaaaaaaaaaaaaaaaaaaaaaaaaaaaaaaaaaaa

SEQ ID NO: 30 - DNA, 40 bp, Artificial sequence
  synthetic
tttttttttttttttttttttttttttttttttttttttt

SEQ ID NO: 31 - DNA, 40 bp, Artificial sequence
  synthetic
actgactgactgactgactgactgactgactgactgactg

SEQ ID NO: 32 - DNA, 40 bp, Artificial sequence
  synthetic
cagtcagtcagtcagtcagtcagtcagtcagtcagtcagt

SEQ ID NO: 33 - DNA, 40 bp, Artificial sequence
  synthetic
tgtgtgtgtgtgtgtgtgtgtgtgtgtgtgtgtgtgtgtg

SEQ ID NO: 34 - DNA, 40 bp, Artificial sequence
  synthetic
cacacacacacacacacacacacacacacacacacacaca

SEQ ID NO: 35 - DNA, 40 bp, Artificial sequence
  synthetic
gtaacgatccagctgtcactgtaacgatccagctgtcact

SEQ ID NO: 36 - DNA, 40 bp, Artificial sequence
  synthetic
agtgacagctggatcgttacagtgacagctggatcgttac

SEQ ID NO: 37 - DNA, 40 bp, Artificial sequence
  synthetic
tcatacgactcactctagggtcatacgactcactctaggg

SEQ ID NO: 38 - DNA, 40 bp, Artificial sequence
  synthetic
ccctagagtgagtcgtatgaccctagagtgagtcgtatga

SEQ ID NO: 39 - DNA, 40 bp, Artificial sequence
  synthetic
actgatggtaatctgcacctactgatggtaatctgcacct

SEQ ID NO: 40 - DNA, 40 bp, Artificial sequence
  synthetic
aggtgcagattaccatcagtaggtgcagattaccatcagt

SEQ ID NO: 41 - DNA, 51 bp, Artificial Sequence
  Synthetic
tctgaagacacatttttactcccaamaagccccctgcagaagtaggagccg